(19) United States
(12) Patent Application Publication
(10) Pub. No.: US 2005/0032724 A1
(43) Pub. Date: Feb. 10, 2005

(54) Methods for improved treatment of cancer with irinotecan based on mrp1

(76) Inventors: Gunther Heinrich, Starnberg (DE); Reinhold Kerb, Munchen (DE)

Correspondence Address: LADAS & PARRY, 26 WEST 61ST STREET, NEW YORK, NY, 10023, US

(21) Appl. No.: 10/484,577

(22) PCT Filed: Jul. 23, 2002
(86) PCT No.: PCT/EP02/08200

(30) Foreign Application Priority Data
Jul. 23, 2001 (EP) 01117608.8
May 24, 2002 (EP) 02011710.7

(51) Int. Cl. A61K 48/00

(52) U.S. Cl. 514/44

(57) ABSTRACT

The present invention relates to the use of irinotecan or derivative thereof for the preparation of a pharmaceutical composition for treating cancer, especially, colorectal cancer, cervical cancer, gastric cancer, lung cancer, malignant glioma, ovarian cancer, and pancreatic cancer in a patient having a genotype with a variant allele which comprises a polynucleotide in accordance with the present invention. Preferably, a nucleotide deletion, addition and/or substitution comprised by said polynucleotide results in an altered expression of a variant allele compared to the corresponding wild type allele or an altered activity of the polypeptide encoded by the variant allele compared to the polypeptide encoded by the corresponding wild type allele. Finally, the present invention relates to a method for selecting a suitable therapy for a subject suffering from colorectal cancer, cervical cancer, gastric cancer, lung cancer, malignant glioma, ovarian cancer, and pancreatic cancer.

The present invention relates to the use of camptothecin drugs, such as irinotecan (CPT-11) or a derivative thereof for the preparation of a pharmaceutical composition for treating cancer, especially, colorectal cancer, cervical cancer, gastric cancer, lung cancer, malignant glioma, ovarian cancer, and pancreatic cancer in a patient having a genotype with a variant allele which comprises a polynucleotide in accordance with the present invention. Preferably, a nucleotide deletion, addition and/or substitution comprised by said polynucleotide results in an altered expression of the variant allele compared to the corresponding wild type allele or an altered activity of the polypeptide encoded by the variant allele compared to the polypeptide encoded by the corresponding wild type allele. Finally, the present invention relates to a method for selecting a suitable therapy for a subject suffering from colorectal cancer, cervical cancer, gastric cancer, lung cancer, malignant glioma, ovarian cancer or pancreatic cancer.

Irinotecan is a semisynthetic analog of the cytotoxic alkaloid camptothecin (CPT), which is obtained from the oriental tree, Camptotheca acuminata Camptothecins demonstrate anti-neoplastic activities by inhibiting specifically with the enzyme topoisomerase I which relieves torsional strain in DNA by inducing reversible single-strand breaks [D'Arpa, et al., 1989, Biochim Biophys Acta 989:163-77, Horwitz, et al., 1973, Cancer Res 33:2834-6]. Irinotecan and its active metabolite SN-38 bind to the topoisomerase I-DNA complex and prevent religation of these single-strand breaks [Kawato, et al., 1991, Cancer Res 51:4187-91]. Irinotecan serves as a water-soluble prodrug of the lipophilic metabolite SN-38 (7-ethyl-10-hydroxycamptothecin) which is formed from irinotecan by carboxylesterase-mediated cleavage of the carbamate bond between the camptothecin moiety and the dipiperidino side chain [Tsuji, et al., 1991, J Pharmacobiodyn 14:341-9]. Carboxylesterase-2 is the primary enzyme involved in this hydrolysis at at pharmacological concentrations [Humerickhouse, et al., 2000, Cancer Res 60:1189-92]. Topoisomerase inhibition and irinotecan-related single strand breaks are caused primarily by SN-38 [Kawato, et al., 1991, Cancer Res 51:4187-91]. Administration of irinotecan has resulted in antitumor activity in mice bearing cancers of rodent origin and in human carcinoma xenografts of various histological types [Furuta, et al., 1988, Gan To Kagaku Ryoho 15:2757-60, Giovanella, et al., 1989, Science 246:1046-8, Giovanella, et al., 1991, Cancer Res 51:3052-5, Hawkins, 1992, Oncology (Huntingt) 6:17-23, Kunimoto, et al., 1987, Cancer Res 47:5944-7].

Irinotecan is also oxidized by CYP3A4 and CYP3A5 [Haaz, et al., 1998, Drug Metab Dispos 26:769-74, Kuhn, 1998, Oncology (Huntingt) 12:39-42, Santos, et al., 2000, Clin Cancer Res 6:2012-20, Rivory, et al., 1996, Cancer Res 56:3689-94]. The major elimination pathway of SN-38 is conjugation with glucuronic acid to form the corresponding glucuronide (SN-38G) [Atsumi, et al., 1991, Xenobiotica 21:1159-69.]. SN-38G is reported to be deconjugated by the intestinal microflora to form SN-38 [Kaneda, et al., 1990, Cancer Res 50:1715-20]. Glucuronidation of SN-38 is mediated by UGT1A1 and UGT1A7 [Lyer, et al., 1998, J Clin Invest 101:847-54, Ciotti, et al., 1999, Biochem Biophys Res Commun 260:199-202]. Mass balance studies have demonstrated that 64% of the total dose is excreted in the feces, confirming the important role of biliary excretion [Slatter, et al., 2000, Drug Metab Dispos 28:423-33]. Studies suggest that the multidrug rsistance protein 1 (MRP1) is a major transporter of irinotecan and its metabolites [Kuhn, 1998, Oncology (Huntingt) 12:39-42, Chen, et al., 1999, Mol Pharmacol 55:921-8, Chu, et al., 1997, Cancer Res 57:1934-8, Chu, et al., 1997, J Pharmacol Exp Ther 281:304-14] and facilitate their biliary excretion, where they cause side effects, although P-glycoprotein also participates in irinotecan excretion [Chu, et al., 1998, Cancer Res 58:5137-43, Chu, et al., 1999, Drug Metab Dispos 27:440-1, Chu, et al., 1999, J Pharmacol Exp Ther 288:735-41, Mattern, et al., 1993, Oncol Res 5:467-74, Hoki, et al., 1997, Cancer Chemother Pharmacol 40:433-8, Sugiyama, et al., 1998, Cancer Chemother Pharmacol 42:S44-9].

Cellular resistance to camptothecins and thus, therapeutic response of irinotecan has been related to intracellular carboxylesterase activity and cleavage activity of topoisomerase I [van Ark-Otte, et al., 1998, Br J Cancer 77:2171-6, Guichard, et al., 1999, Br J Cancer 80:364-70].

The use of such camptothecin drugs, e.g. irinotecan, is limited by clearly dose-dependent myelosuppression and gastrointestinal toxicities, including nausea, vomiting, abdominal pain, and diarrhea which side effects can prove fatal. The major dose-limiting toxicity of irinotecan therapy is diarrhea, which occurs in up to 88% of patients and which depends on intestinal SN-38 accumulation [van Ark-Otte, et al., 1998, Br J Cancer 77:2171-6, Guichard, et al., 1999, Br J Cancer 80:364-70, Araki, et al., 1993, Jpn J Cancer Res 84:697-702] secondary to the biliary excretion of SN-38, the extent of which is determined by SN-38 glucuronidation [Gupta, et al., 1994, Cancer Res 54:3723-5, Gupta, et al., 1997, J Clin Oncol 15:1502-10]. Myelosuppression has been correlated with the area under the concentration-time curve of both irinotecan and SN-38 [Sasaki, et al., 1995, Jpn J Cancer Res 86:101-10].

Despite the approval of irinotecan for patients with metastatic colorectal cancer refractory to 5-fluorouracil therapy in 1997, the therapeutic benefit remains questionable. Recently two large clinical trials on colorectal cancer involving more than 2000 patients had to be canceled by the National Institute of Cancer (NCI) due to an almost 3-times increase of irinotecan toxicity-related mortality within the first 60 days of treatment. Causes of death were diarrhea- and vomiting-related dehydratation and neutropenia-related sepsis [2001, arznei-telegramm 32:58]. Although irinotecan was proven to be effective against thencancer itself, not all patients could benefit from longterm survival due to short term toxicity. Thus, it is highly desirable to identify those patients who will most likely suffer from irinotecan toxicity.

Currently, patients are treated according to most treatment schedules with a standard dose of initially 60 to 125 mg/m² irinotecan in combination with other anti-neoplastic drugs administered several courses of 3 to 4 weekly dosings, and subsequent doses are adjusted in 25 to 50 mg/m² increments based upon individual patient tolerance to treatment. Treatment may be delayed 1 to 2 weeks to allow for recovery from irinotecan-related toxicity and if the patient has not recovered, therapy has to be discontinued. Provided intolerable toxicity does not develop, treatment with additional courses are continued indefinitely as long as the patient continues to experience clinical benefit. Response rates varies depending from tumor type from less than 10% to almost 90%. However, it takes at least 6 to 8 weeks to evaluate therapeutic response and to consider altematives. Thus, finding the right dosage for the patient is tedious, time-consuming and takes the risk of lifethreatening adverse effects. Patients might be unnecessarily put to this risk who do not benefit from treatment and additionally, worthwhile time is wasted before these patients receive their suitable treatment.

Furthermore, as observed for many chemotherapeutic agents, the risk to develop cellular resistances against therapy is increased upon suboptimal exposure of cells to chemotherapeutic agents, such as irinotecan.

Pharmacokinetic modulation with inhibitors of biliary excretion (e. g., MRP and P-glycoprotein) and inducers of UGT1A1 have been suggested as a tool to reduce camptothecin-related toxicity [Gupta, et al., 1996, Cancer Res 56:1309-14, Gupta, et al., 1997, Cancer Chemother Pharmacol 39:440-4]. Although preliminary data of a clinical study of irinotecan in combination with cyclosporine A, and phenobarbital show some promising results in respect to limit camptothecin-related diarrhea [Ratain, 2000, Clin Cancer Res 6:3393-4], cotreatment with drugs such as cyclosporine A, and phenobarbital takes the additional risk of adverse events and drug interactions.

Large interpatient variability exist for both SN-38 and SN-38G pharmacokinetics [Canal, et al., 1996, J Clin Oncol 14:2688-95], which is likely to be due to interpatient differences in the metabolism pathways of irinotecan [Rivory, et al., 1997, Clin Cancer Res 3:1261-6]. Furthermore, severe irinotecan toxicity has been reported in patients with Gilbert syndrome [Wasserman, et al., 1997, Ann Oncol 8:1049-51]. Consequently, a genetic predisposition to the metabolism of irinotecan, that patients with low UGT1A1 activity are at increased risk for irinotecan toxicity has been suggested [lyer, et al., 1998, J Clin Invest 101:847-54, Ando, et al., 1998, Ann Oncol 9:845-7]. A common polymorphism in the UGT1A1 promoter [Monaghan, et al., 1996, Lancet 347:578-81] has been correlated with in vitro glucuronidation of SN-38 [lyer, et al., 1999, Clin Pharmacol Ther 65:576-82], and its possible clinical use has been suggested from a case control study [Ando, et al., 2000, Cancer Res 60:6921-6]. However, irinotecan-related toxicity was predicted by UGT1A1 genotype only in the minority of affected patients (<15%).

In conclusion, it would be highly desirable to significantly improve therapeutic efficacy and safety of camptothecin-based therapies and to avoid therapy-caused fatalities, to avoid unnecessary development of resistances, and to reduce adverse events- and therapeutic delay-related hospitalization costs. However, no accepted mechanism for reducing irinotecan toxicity or to improve therapeutic efficacy are currently available.

Thus, the technical problem underlying the present invention is to provide improved means and methods for the efficient treatment of colorectal cancer, cervical cancer, gastric cancer, lung cancer, malignant glioma, ovarian cancer, and pancreatic cancer, whereby the aforementioned undesirable side effects are to be avoided. The technical problem underlying the present invention is solved by the embodiments characterized in the claims.

Accordingly, the present invention relates to the use of irinotecan or a derivative thereof for the preparation of a pharmaceutical composition for treating cancer, especially, colorectal cancer, cervical cancer, gastric cancer, lung cancer, malignant glioma, ovarian cancer, and pancreatic cancer in a subject having a genome with a variant allele which comprises a polynucleotide selected from the group consisting of:
(a) a polynucleotide having the nucleic acid sequence of any one of SEQ ID NOs: 169, 170, 173, 174, 177, 178, 181, 182, 185, 186, 189, 190, 193, 194, 197, 198, 201, 202, 205, 206, 209, 210, 213, 214, 217, 218, 221, 222, 225, 226, 229, 230, 233, 234, 237, 238, 241, 242, 245, 246, 249, 250, 253, 254, 257, 258, 261, 262, 265, 266, 269, 270, 273, 274, 277, 278, 281, 282, 285, 286, 289, 290, 293, 294, 297, 298, 301, 302, 305, 306, 309, 310, 313, 314, 317, 318, 321, 322, 325, 326, 329, 330, 333 and/or 334;
(b) a polynucleotide encoding a polypeptide having the amino acid sequence of any one of SEQ ID NOs: 600, 602 and/or 604;
(c) a polynucleotide capable of hybridizing to a Multidrug Resistance Protein 1 (MRP1) gene, wherein said polynucleotide is having at a position corresponding to positions 57998, 57853, 53282, and/or 39508 of the MRP1 gene (Accession No: GI:7209451), a substitution or deletion of at least one nucleotide or at a position corresponding to positions 137667, 137647, 137710, 124667, and/or 38646 of the MRP1 gene (Accession No: AC026452), a substitution or deletion of at least one nucleotide or at a position corresponding to positions 27258, 27159, 34218, 34215, 55472, and/or 34206 to 34207 of the MRP1 gene (Accession No: AC003026), a substitution or deletion of at least one nucleotide or at a position corresponding to positions 21133, 14008, 18067, 17970, and/or 17900 of the MRP1 gene (Accession No: U91318), a substitution or deletion of at least one nucleotide or at a position corresponding to positions 79, 88, and/or 249 of the MRP1 gene (Accession No: AF022830), a substitution or deletion of at least one nucleotide or at a position corresponding to positions 95 and/or 259 of the MRP1 gene (Accession No: AF022831), a substitution or deletion of at least one nucleotide or at a position corresponding to positions 150727 and/or 33551 of the MRP1 gene (Accession No: AC025277), a substitution or deletion of at least one nucleotide or at a position corresponding to position 174 of the MRP1 gene (Accession No: AF022828), a substitution or deletion of at least one nucleotide or at a position corresponding to positions 248 and/or 258 of the MRP1 gene (Accession No: AF022829), a substitution or deletion of at least one nucleotide or at a position corresponding to positions 1884, 1625, 1163, 381, 233, 189, 440, and/or 1720 to 1723 of the MRP1 gene (Accession No: U07050), a substitution or deletion of at least one nucleotide or at a position corresponding to positions 926/927 and/or 437/438 of the MRP1 gene (Accession No: U07050) a insertion of at least one nucleotide or at a position corresponding to position 55156/55157 of the MRP1 gene (Accession No: AC003026) a insertion of at least one nucleotide;
(d) a polynucleotide capable of hybridizing to a MRP1 gene, wherein said polynucleotide is having at a position corresponding to position 21133, 14008 and/or 18195 of the MRP1 gene (Accession No: U91318) or at a position corresponding to position 27258 and/or 34218 of the MRP1 gene (Accession No: AC003026) or at a position corresponding to position 79 of the MRP1 gene (Accession No: AF022830) or at a position corresponding to position 57998, and/or 57853 of the MRP1 gene (Accession No: GI:7209451) or at a position corresponding to position 137667 and/or 137647 of the MRP1 gene (Accession No: AC026452) or at a position corresponding to position 150727 and/or 33551 of the MRP1 gene (Accession No: AC025277) or at a position corresponding to position 248 of the MRP1 gene (Accession No: AF022829) or at a position corresponding to position 1884, 1625, 233, and/or 189 of the MRP1 gene (Accession No: U07050) an A, at a position corresponding to position 39508 of the MRP1 gene (Accession No: GI:7209451) or at a position corresponding to position 17900, 18067 and/or 18195 of the MRP1 gene (Accession No: U91318) or at a position corresponding to position 174 of the MRP1 gene (Accession No: AF022828) or at a position corresponding to position 440 and/or 1163 of the MRP1 gene (Accession No: U07050) a T, at a position corresponding to position 88 of the MRP1 gene (Accession No: AF022830) or at a position corresponding to position 95 of the MRP1 gene (Accession No: AF022831) or at a position corresponding to position 27159, 55472 and/or 34215 of the MRP1 gene (Accession No: AC003026) or at a position corresponding to position 124667 and/or 38646 of the MRP1 gene (Accession No: AC026452) or at a position corresponding to position 53282 of the MRP1 gene (Accession No: GI:7209451) or at a position corresponding to position 137710 of the MRP1 gene (Accession No: AC026452) a C, at a position corresponding to position 249 of the MRP1 gene (Accession No: AF022830) or at a position corresponding to position 258 of the MRP1 gene (Accession No: AF022829) or at a position corresponding to position 259 of the MRP1 gene (Accession No: AF022831) or at a position corresponding to position 381 of the MRP1 gene (Accession No: U07050) a G, at a position corresponding to position 17970 of the MRP1 gene (Accession No: U91318) a deletion of a T or at a position corresponding to position 34206 to 34207 of the MRP1 gene (Accession No: AC003026) a deletion of a AT or at a position corresponding to position 1720 to 1723 of the MRP1 gene (Accession No: U07050) a deletion of GGTA, at a position corresponding to position 926/927 a insertion of a T and/or 437/438 of the MRP1 gene (Accession No: U07050) a insertion of a TCCTTCC, at a position corresponding to position 55156/55157 of the MRP1 gene (Accession No: AC003026) a insertion of TGGGGC;
(e) a polynucleotide encoding an MRP1 polypeptide or fragment thereof, wherein said polypeptide comprises an amino acid substitution of Phe to Cys at a position corresponding to position 239 of the MRP1 polypeptide (Accession No: G2828206) or/and Arg to Ser at a position corresponding to position 433 of the MRP1 polypeptide (Accession No: G2828206) or/and Arg to Gin at a position corresponding to position 723 of the MRP1 polypeptide (Accession No: G2828206).

The term “irinotecan or a derivative thereof” as used in accordance with the present invention preferably refers to a substance which is characterized by the general structural formula 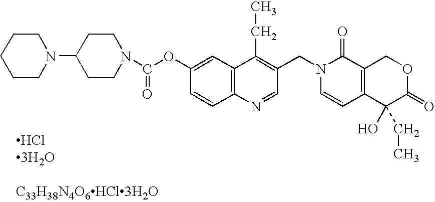
further described in U.S. Pat. No. 05,106,742, U.S. Pat. No. 05,340,817, U.S. Pat. No. 05,364,858, U.S. Pat. No. 05,401,747, U.S. Pat. No. 05,468,754, U.S. Pat. No. 05,559,235 and U.S. Pat. No. 05,663,177. Moreover, also comprised by the term “irinotecan or a derivative thereof” are analogues and derivatives of camptothecin. The types and ranges of camptothecin analogues available are well known to those of skill in the art and described in numerous texts, e.g. [Hawkins, 1992, Oncology (Huntingt) 6:17-23, Burris, et al., 1994, Hematol Oncol Clin North Am 8:333-55, Slichenmyer, et al., 1993, J Natl Cancer Inst 85:271 -91, Slichenmyer, et al., 1994, Cancer Chemother Pharmacol 34:S53-7]. Specific examples of active camptothecin analogues are hexacyclic camptothecin analogues, 9-nitro-camptothecin, camptothecin analogues with 20S configuration with 9- or 10-substituted amino, halogen, or hydroxyl groups, seven-substituted water-soluble camptothecins, 9-substituted camptothecins, E-ring-modified camptothecins such as (RS)-20-deoxyamino-7-ethyl-10-methoxycamptothecin, and 10-substituted camptothecin analogues [Emerson, et al., 1995, Cancer Res 55:603-9, Ejima, et al., 1992, Chem Pharm Bull (Tokyo) 40:683-8, Sugimori, et al., 1994, J Med Chem 37:3033-9, Wall, et al., 1993, J Med Chem 36:2689-700, Wani, et al., 1980, J Med Chem 23:554-60, Kingsbury, et al., 1991, J Med Chem 34:98-107]. Various other camptothecin analogues with similar therapeutic activity are described [Hawkins, 1992, Oncology (Huntingt) 6:17-23, Burris and Fields, 1994, Hematol Oncol Clin North Am 8:333-55, Slichenmyer, et al., 1993, J Natl Cancer Inst 85:271-91, Slichenmyer, et al., 1994, Cancer Chemother Pharmacol 34:S53-7]. Suitable methods for synthezising camptothecin analogues are described [Emerson, et al., 1995, Cancer Res 55:603-9, Ejima, et al., 1992, Chem Pharm Bull (Tokyo) 40:683-8, Sugimori, et al., 1994, J Med Chem 37:3033-9, Wall, et al., 1993, J Med Chem 36:2689-700, Wani, et al., 1980, J Med Chem 23:554-60, Kingsbury, et al., 1991, J Med Chem 34:98-107, Sugasawa, et al., 1976, J Med Chem 19:675-9].

Said substances are known to be therapeutically useful as described, e.g., in colorectal cancer, non-small cell and small cell lung cancer, oesophageal cancer, renal cell carcinoma, ovarian cancer, breast cancer, pancreatic cancer, squamous cell cancer, leukemias and lymphomas [Kawato, et al., 1991, Cancer Res 51:4187-91, Furuta, et al., 1988, Gan To Kagaku Ryoho 15:2757-60, Hawkins, 1992, Oncology (Huntingt) 6:17-23, Slichenmyer, et al., 1993, J Natl Cancer Inst 85:271-91, Slichenmyer, et al., 1994, Cancer Chemother Pharmacol 34:S53-7, Tsuruo, et al., 1988, Cancer Chemother Pharmacol 21:71-4, Wiseman, et al., 1996, Drugs 52:606-23, Gottlieb, et al., 1970, Cancer Chemother Rep 54:461-70, Negoro, et al., 1991, J Natl Cancer Inst 83:1164-8, Rowinsky, et al., 1994, Cancer Res 54:427-36]. Also encompassed by the use of the present invention are derivatives of those substances which are obtainable by way of any chemical modification, wherein said derivatives are equally well therapeutically suited for the use of the present invention. To determine whether a derivative of the substances of the invention is equally well therapeutically suited for the use of the invention biological assays well known in the art can be performed. Such assays are described, e.g., in [Kawato, et al., 1991, Cancer Res 51:4187-91, Furuta, et al., 1988, Gan To Kagaku Ryoho 15:2757-60, Giovanella, et al., 1989, Science 246:1046-8, Giovanella, et al., 1991, Cancer Res 51:3052-5, Kunimoto, et al., 1987, Cancer Res 47:5944-7, Mattern, et al., 1993, Oncol Res 5:467-74, Tsuruo, et al., 1988, Cancer Chemother Pharmacol 21:71-4, Burris, et al., 1992, J Natl Cancer Inst 84:1816-20, Friedman, et al., 1994, Cancer Chemother Pharmacol 34:171-4].

It is contemplated that any of the compounds described in the above publications may be used in this invention.

It has been show that irinotecan is particularly well suited for the treatment of colorectal cancer, cervical cancer, gastric cancer, lung cancer, malignant glioma, ovarian cancer, and pancreatic cancer. Thus, most preferably the substance used according to the present invention is irinotecan.

The term “pharmaceutical composition” as used herein comprises the substances of the present invention and optionally one or more pharmaceutically acceptable carrier. The substances of the present invention may be formulated as pharmaceutically acceptable salts. Acceptable salts comprise acetate, methylester, HCl, sulfate, chloride and the like. The pharmaceutical compositions can be conveniently administered by any of the routes conventionally used for drug administration, for instance, orally, topically, parenterally or by inhalation. The substances may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. The substance according to the present invention can be administered in various manners to achieve the desired effect. Said substance can be administered either alone or in the formulated as pharmaceutical preparations to the subject being treated either orally, topically, parenterally or by inhalation. Moreover, the substance can be administered in combination with other substances either in a common pharmaceutical composition or as separated pharmaceutical compositions.

The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of the substance according to the invention which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

A typical dose can be, for example, in the range of 5 to 100 mg however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 1 mg per m² body surface to about 500 mg per m² body surface, usually 20 to 200 mg per m² body surface.

The pharmaceutical compositions and formulations referred to herein are administered at least once in accordance with the use of the present invention. However, the said pharmaceutical compositions and formulations may be administered more than one time, for example once weekly every other week up to a non-limited number of weeks.

Specific formulations of the substance according to the invention are prepared in a manner well known,in the pharmaceutical art and usually comprise at least one active substance referred to herein above in admixture or otherwise associated with a pharmaceutically acceptable carrier or diluent thereof. For making those formulations the active substance(s) will usually be mixed with a carrier or diluted by a diluent, or enclosed or encapsulated in a capsule, sachet, cachet, paper or other suitable containers or vehicles. A carrier may be solid, semisolid, gel-based or liquid material which serves as a vehicle, excipient or medium for the active ingredients. Said suitable carriers comprise those mentioned above and others well known in the art, see, e.g., Remington's Pharmaceutical Sciences, Mack Publishing Company, Easton, Penn. The formulations can be adopted to the mode of administration comprising the forms of tablets, capsules, suppositories, solutions, suspensions or the like.

The dosing recommendations will be indicated in product labeling by allowing the prescriber to anticipate dose adjustments depending on the considered patient group, with information that avoids prescribing the wrong drug to the wrong patients at the wrong dose.

The term “treating” means alleviation of the diseases symptoms, i.e., regression of symptoms or inhibited progression of such symptoms, in subjects or disease populations which have been treated. Said alleviation of the diseases can be monitored by the degree of the clinical symptoms (e.g. tumor size) accompanied with the disease. While the invention may not be effective in 100% of patients treated, it is effective in treating a statistically significant (p value less than 0.05) number of patients. Whether said number of subjects is significant can be determined by statistical tests such as the Student's t-test, the chi²-test, the U-test according to Mann and Whitney, the Kruskal-Wallis-test (H-Test), Jonckheere-Terpstra-test or the Wilcoxon-test.

The present invention also encompasses all embodiments described in connection with pharmaceutical compositions in U.S. Pat. No. 05,106,742, U.S. Pat. No. 05,340,817, U.S. Pat. No. 05364858, U.S. Pat. No. 05401747, U.S. Pat. No. 05468754, U.S. Pat. No. 05559235 and U.S. Pat. No. 05,663,177.

The terms “colorectal cancer, cervical cancer, gastric cancer, lung cancer, malignant glioma, ovarian cancer, and pancreatic cancer” comprise diseases and dysregulations related to cancer. Preferred diseases encompassed by the use of the present invention are colorectal cancer, cervical cancer, gastric cancer, lung cancer, malignant glioma, ovarian cancer, and pancreatic cancer. Said diseases and dysregulations are well known in the art and the accompanied symptoms are described, e.g., in standard text books such as Stedman.

The term “subject” as used in the sense of the present invention comprises animals, preferably those specified herein after, and humans.

The term “variant allele” as used herein refers to a polynucleotide comprising one or more of the polynucleotides described herein below corresponding to a MRP1 gene. Each individual subject carries at least two alleles of the MRP1 gene, wherein said alleles are distinguishable or identical. In accordance with the use of the present invention a variant allele comprises at least one or more of the polynucleotides specified herein below. Said polynucleotides may have a synergistic influence on the regulation or function of the first variant allele. Preferably, a variant allele in accordance with the use of the present invention comprises at least two of the polynucleotides specified herein.

In the context of the present invention the term “polynucleotides” or “polypeptides” refers to different variants of a polynucleotide or a polypeptide specified in accordance with the uses of the present invention. Said variants comprise a reference or wild type sequence of the polynucleotides or polypeptides specified herein as well as variants which differ therefrom in structure or composition. Reference or wild type sequences for the polynucleotides are Genbank accession No: GI:8850235, GI:11118740, GI:10281451, GI:11177452, GI:10281451, GI:6706037, U91318, GI:7209451, AC026452, AC003026, U91318, AF022830, GI:7209451, AC026452, AC003026, AC025277, AF022828, AF022829, AF022831, U07050, AC003026, AC002457, AC005068, M29432, M29445, and GI:11225259 or Accession No (Pid No): G8850236, G2828206, G2506118, and G12644118 for polypeptides. The differences in structure or composition usually occur by way of nucleotide or amino acid substitution(s), addition(s) and/or deletion(s).

Preferably, said nucleotide substitution(s), addition(s) or deletion(s) referred to in accordance with the use of the present invention result(s) in one or more changes of the corresponding amino acid(s) of the polypeptides. The variant polynucleotides also comprise fragments of said polynucleotides or polypeptides. The polynucleotides or polypeptides as well as the aforementioned fragments thereof are characterized as being associated with a MRP1 dysfunction or dysregulation comprising, e.g., insufficient and/or altered drug uptake. Preferred deletions in accordance with the invention are a T or AT deletion at a position corresponding to position 17970 of the MRP1 gene (Accession No: U91318) and/or 34206 to 34207 of the MRP1 gene (Accession No: AC003026), preferred insertion is a TCCTTCC at a position corresponding to position 437/438 of the MRP1 gene (Accession No: GI: U07050) and/or a TGGGGC insertion at a position corresponding to position 55156/55157 of the MRP1 gene (Accession No: AC003026).

The present invention also encompasses all embodiments described in connection with polynucleotides in WO9957322, WO0109183 or U.S. Pat. No. 5,786,344.

The term “hybridizing” as used herein refers to polynucleotides which are capable of hybridizing to the above polynucleotides or parts thereof which are associated with a MRP1 dysfunction or dysregulation. Thus, said hybridizing polynucleotides are also associated with said dysfunctions and dysregulations. Preferably, said polynucleotides capable of hybridizing to the aforementioned polynucleotides or parts thereof which are associated with MRP1 dysfunctions or dysregulations are at least 70%, at least 80%, at least 95% or at least 100% identical to the polynucleotides or parts thereof which are associated with MRP1 dysfunctions or dysregulations. Therefore, said polynucleotides may be useful as probes in Northern or Southern Blot analysis of RNA or DNA preparations, respectively, or can be used as oligonucleotide primers in PCR analysis dependent on their respective size. Also comprised in accordance with the use of the invention are hybridizing polynucleotides which are useful for analyzing DNA-Protein interactions via, e.g., electrophoretic mobility shift analysis (EMSA). Preferably, said hybridizing polynucleotides comprise at least 10, more preferably at least 15 nucleotides in length while a hybridizing polynucleotide to be used as a probe preferably comprises at least 100, more preferably at least 200, or most preferably at least 500 nucleotides in length.

It is well known in the art how to perform hybridization experiments with nucleic acid molecules, i.e. the person skilled in the art knows what hybridization conditions s/he has to use in accordance with the present invention. Such hybridization conditions are referred to in standard text books, such as Molecular Cloning, A Laboratory Manual, Cold Spring Harbor Laboratory (1989) N.Y. Preferred in accordance with the use of the present inventions are polynucleotides which are capable of hybridizing to the above polynucleotides or parts thereof which are associated with a MRP1 dysfunction or dysregulation under stringent hybridization conditions, i.e. which do not cross hybridize to unrelated polynucleotides such as polynucleotides encoding a polypeptide different from the MRP1 polypeptides of the invention.

Moreover, methods for determining whether a subject comprises a polynucleotide referred to herein above are well known in the art. To carry out said methods, it might be necessary to take a sample comprising biological material, such as isolated cells or tissue, from said subject. Further, the methods known in the art could comprise for example, PCR based techniques, RFLP-based techniques, DNA sequencing-based techniques, hybridization techniques, Single strand conformational polymorphism (SSCP), denaturating gradient gel electrophoresis (DGGE), mismatch cleavage detection, heteroduplex analysis, techniques based on mass spectroscopy, HPLC-based techniques, primer extension-based techniques, and 5′-nuclease assay-based techniques. A preferred and convenient method to be used in order to determine the presence or absence of one or more of the above specified polynucleotides is to isolate blood cells from a subject and to perform a PCR based assay on genomic DNA isolated from those blood cells, whereby the PCR is used to determine whether said polynucleotides specified herein above or parts thereof are present or absent. Said method is described in more detail below and in the Examples.

The term “corresponding” as used herein means that a position is not only determined by the number of the preceding nucleotides and amino acids, respectively. The position of a given nucleotide or amino acid in accordance with the use of the present invention which may be deleted, substituted or comprise one or more additional nucleotide(s) may vary due to deletions or additional nucleotides or amino acids elsewhere in the gene or the polypeptide. Thus, under a “corresponding position” in accordance with the present invention it is to be understood that nucleotides or amino acids may differ in the indicated number but may still have similar neighboring nucleotides or amino acids. Said nucleotides or amino acids which may be exchanged, deleted or comprise additional nucleotides or amino acids are also comprised by the term “corresponding position”. Said nucleotides or amino acids may for instance together with their neighbors form sequences which may be involved in the regulation of gene expression, stability of the corresponding RNA or RNA editing, as well as encode functional domains or motifs of the protein of the invention.

By, e.g., “position 17970 to 17970” it is meant that said polynucleotide comprises one or more deleted nucleotides which are deleted between positions 17970 and position 17970 of the corresponding wild type version of said polynucleotide. The same applies mutatis mutandis to all other position numbers referred to in the above embodiment which are drafted in the same format.

By, e.g., “position 1222/1223” it is meant that said polynucleotide comprises one or more additional nucleotide(s) which are inserted between positions 1222 and position 1223 of the corresponding wild type version of said polynucleotide. The same applies mutatis mutandis to all other position numbers referred to in the above embodiment which are drafted in the same format, i.e. two consecutive position numbers separated by a slash (/).

In accordance with the present invention, the mode and population distribution of genetic variations in the MRP1 gene—the different alleles of the MRP1 gene—have been analyzed by sequence analysis of relevant regions of the human said gene from many different individuals. It is a well known fact that genomic DNA of individuals, which harbor the individual genetic makeup of all genes, including the MRP1 gene, can easily be purified from individual blood samples. These individual DNA samples are then used for the analysis of the sequence composition of the alleles of the MRP1 gene that are present in the individual which provided the blood sample. The sequence analysis was carried out by PCR amplification of relevant regions of said genes, subsequent purification of the PCR products, followed by automated DNA sequencing with established methods (e.g. ABI dyeterminator cycle sequencing).

One important parameter that has to be considered in the attempt to determine the individual genotypes and identify novel variants of the MRP1 gene by direct DNA-sequencing of PCR-products from human blood genomic DNA is the fact that each human harbors (usually, with very few abnormal exceptions) two gene copies of each autosomal gene (diploidy). Because of that, great care has to be taken in the evaluation of the sequences to be able to identify unambiguously not only homozygous sequence variations but also heterozygous variations. The details of the different steps in the identification and characterization of the polymorphisms in the MRP1 gene (homozygous and heterozygous) are described in the Examples below.

Over the past 20 years, genetic heterogeneity has been increasingly recognized as a significant source of variation in drug response. Many scientific communications (Meyer, Ann. Rev. Pharmacol. Toxicol. 37 (1997), 269-296 and West, J. Clin. Pharmacol. 37 (1997), 635-648) have clearly shown that some drugs work better in some patients than in others or may even be highly toxic and that such variations in patients' responses to drugs can be correlated to a molecular basis. This “pharmacogenomic” concept spots correlations between responses to drugs and genetic profiles of patient's (Marshall, Nature Biotechnology, 15 (1997), 954-957; Marshall, Nature Biotechnology, 15 (1997), 1249-1252). In this context of population variability with regard to drug therapy, pharmacogenomics has been proposed as a tool useful in the identification and selection of patients which can respond to a particular drug without side effects. This identification/selection can be based upon molecular diagnosis of genetic polymorphisms by genotyping DNA from leukocytes in the blood of a patient, for example, and characterization of disease (Bertz, Clin. Pharmacokinet. 32 (1997), 210-256; Engel, J. Chromatogra. B. Biomed. Appl. 678 (1996), 93-103). For the founders of health care, such as health maintenance organizations in the US and government public health services in many European countries, this pharmacogenomics approach can represent a way of both improving health care and reducing costs related to health care caused by the development of unnecessary drugs, by ineffective drugs and by side effects due to drug administration.

The mutations in the variant genes of the invention sometimes result in amino acid deletion(s), insertion(s) and in particular in substitution(s) either alone or in combination. It is of course also possible to genetically engineer such mutations in wild type genes or other mutant forms. Methods for introducing such modifications in the DNA sequence of said genes are well known to the person skilled in the art; see, e.g., Sambrook, Molecular Cloning A Laboratory Manual, Cold Spring Harbor Laboratory (1989) N.Y.

For the investigation of the nature of the alterations in the amino acid sequence of the polypeptides of the invention may be used such as BRASMOL that are obtainable from the Internet. Furthermore, folding simulations and computer redesign of structural motifs can be performed using other appropriate computer programs (Olszewski, Proteins 25 (1996), 286-299; Hoffman, Comput. Appl. Biosci. 11 (1995), 675-679). Computers can be used for the conformational and energetic analysis of detailed protein models (Monge, J. Mol. Biol. 247 (1995), 995-1012; Renouf, Adv. Exp. Med. Biol. 376 (1995), 37-45). These analysis can be used for the identification of the influence of a particular mutation on metabolism, binding, inhibition, mediating of therapeutic action and/or transport of drugs. Moreover, based on the knowledge of the altered structure of the polypeptides which are encoded by the polynucleotides specified in the use of the present invention derivatives of the substances referred to above can be designed and synthesized which can be more efficiently metabolized, modified, transported, eliminated, and/or binded. Thereby, drugs or pro-drugs can be designed on the basis of the substances referred to herein which are more efficient in therapy of colorectal cancer, cervical cancer, gastric cancer, lung cancer, malignant glioma, ovarian cancer, and pancreatic cancer in a subject having a genotype characterized by the presence of one or more polynucleotides of the invention.

Usually, said amino acid deletion, addition or substitution in the amino acid sequence of the protein encoded by the polynucleotide referred to in accordance with the use of the present invention is due to one or more nucleotide substitution, insertion or deletion, or any combinations thereof. Preferably said nucleotide substitution, insertion or deletion may result in an amino acid substitution of the MRP1 gene are Phe to Cys at a position corresponding to position 329, Arg to Ser at a position corresponding to position 433 and/or Arg to Glu at a position corresponding to position 723 of the MRP1 polypeptide (Accession No:62828206). The polypeptides encoded by the polynucleotides referred to in accordance with the use described herein have altered biological properties due to the mutations referred to in accordance with the present invention. Examples for said altered properties are stability of the polypeptides or amount of the polypeptides which may be effected resulting in, e.g. an altered drug metabolism or an altered transport of drugs or an altered substrate specificity or an altered catalytic activity characterized by, e.g. insufficiencies in drug metabolism or a complete loss of the capability to metabolize drugs or an enhanced capacity to metabolize drugs or an altered transport activity characterized by, e.g., insufficiencies in drug transport or a complete loss of the capability of transporting drugs or an altered substrate binding characterized by, e.g. an altered drug action or an altered inhibition or induction of transport or an altered binding to receptors or other target molecules characterized by, e.g. an altered activation of signal transduction pathways or an altered protein or enzyme function. These altered properties result in an impaired pharmacological response to the substances referred to above of the subject to be treated in accordance with the use of the present invention. Moreover, due to said altered properties of the polypeptides encoded by the variant alleles specified herein the substances may be chemically modified in a way resulting in derivatives of the substances which are harmful or toxic for the subject or which cause undesirable side effects.

The mutations in the MRP1 gene detected in accordance with the present invention are listed in Tables 1 and 2. As is evident to the person skilled in the art, the genetic knowledge of the polynucleotides specified herein above can be used to exactly and reliably characterize the genotype of a patient.

Advantageously, therapeutical measures which are based on irinotecan or a derivative thereof can be more efficiently applied when taking into consideration said genetic knowledge. Undesirable side effects of said substances can be avoided and an effective but not harmful dosage can be calculated individually due the knowledge of the genetic makeup of the subject. Moreover in accordance with the foregoing, in cases where a given drug causes an unusual effect, a suitable individual therapy can be designed based on the knowledge of the individual genetic makeup of a subject. This tailored therapy will also be suitable to avoid the occurance of therapy resistances. Said resistances are one major problem in cancer chemotherapy with various chemotherapeutic agents, this fact being well known in the art. The use of the present invention, therefore, provides an improvement of the therapeutic applications which are based on the known therapeutically desirable effects of the substances referred to herein above since it is possible to individually treat the subject with an appropriate dosage and/or an appropriate derivative of said substances. Thereby, undesirable, harmful or toxic effects are efficiently avoided. Furthermore, the use of the present invention provides an improvement of the therapeutic applications which are based on the known therapeutically desirable effects of the substances referred to herein above since it is possible to identify those subject prior to onset of drug therapy and treat only those subjects with an appropriate dosage and/or an appropriate derivative of said substances who are most likely to benefit from therapy with said substances. Thereby, the unnecessary and potentially harmful treatment of those subjects who do not respond to the treatment with said substances (nonresponders), as well as the development of drug resistances due to suboptimal drug dosing can be avoided.

In accordance with the present invention it has been surprisingly found that a variant allele corresponding to the MRP1 gene alters the pharmacological response of said subject to the administration of irinotecan or a derivative thereof. Hence, in accordance with the use of the present invention the diseases and disorders referred to herein can be more efficiently treated whereby said therapies measures are more convenient for the subject. Moreover, the applicability of therapeutic measures comprising administration of the substances referred to herein above can be efficiently predicted.

In a preferred embodiment of the use of the present invention said variant allele comprises a polynucleotide selected from the group consisting of:
(a) a polynucleotide having the nucleic acid sequence of any one of SEQ ID NO: 181, 209, 217, 205, 277, 281, 301, 325, 229,193, 313, 293 or 253:
(b) a polynucleotid encoding a polypeptide having the amino acid sequence of SEQ ID NO: 600;
(c) a polynucleotide capable of hybridizing to a MRP1 gene, wherein said polynucleotide is having a substitution at a position corresponding to position 137647 of the MRP1 gene (Accession No: AC026452), 95 of the MRP1 gene (Accession No: AF022831), 53282 of the MRP1 gene (Accession No: GI:7209451), 249 of the MRP1 gene (Accession No: AF022830), 259 of the MRP1 gene (Accession No: AF022831), 124667 of the MRP1 gene (Accession No: AC026452), 381, 440,1625 of the MRP1 gene (Accession No: U07050), 34218 of the MRP1 gene (Accession No: AC003026), 18067 or 17900 of the MRP1 gene (Accession No: U91318) or an insertion of at least one nucleotide at a position corresponding to position 926/927 of the MRP1 gene (Accession No: U07050);
(d) a polynucleotide capable of hybridizing to a MRP1 gene, wherein said polynucleotide is having a T at a position corresponding to position 137647 of the MRP1 gene (Accession No: AC026452), 18067 or 17900 of the MRP1 gene (Accession No: U91318), 440 of the MRP1 gene (Accession No: U07050), a C at a position corresponding toposition 95 of the MRP1 gene (Accession No: AF022831), 124667 of the MRP1 gene (Accession No: AC026452), a G at a position corresponding to position 53282 of the MRP1 gene (Accession No: GI:7209451), 249 of the MRP1 gene (Accession No: AF022830), 259 of the MRP1 gene (Accession No: AF022831), 381 of the MRP1 gene (Accession No: U07050), or an A at a position corresponding to position 34218 of the MRP1 gene (Accession No: AC003026) or 1625 of the MRP1 gene (Accession No: U07050) or an insertion of a T at a position corresponding to position 926/927 of the MRP1 gene (Accession No: U07050);
(e) a polynucleotide encoding an MRP1 polypeptide or fragment thereof, wherein said polypeptide comprises an amino acid substitution at a position corresponding to position 329 of the MRP1 polypeptide (Accession No: G2828206); and
(f) a polynucleotide encoding an MRP1 polypeptide or fragment thereof, wherein said polypeptide comprises an amino acid substitution of Phe to Cys at a position corresponding to position 329 of the MRP1 polypeptide (Accession No: G2828206).

More preferably, said variant allele comprises a polynucleotide selected from the group consisting of:
(a) a polynucleotide having the nucleic acid sequence of any one of SEQ ID NO: 209, 205, 277, 281, 301 or 325;
(b) a polynucleotid encoding a polypeptide having the amino acid sequence of SEQ ID NO: 600;
(c) a polynucleotide capable of hybridizing to a MRP1 gene, wherein said polynucleotide is having a substitution at a position corresponding to position 95 of the MRP1 gene (Accession No: AF022831), 249 of the MRP1 gene (Accession No: AF022830), 259 of the MRP1 gene (Accession No: AF022831), 124667 of the MRP1 gene (Accession No: AC026452), 381 of the MRP1 gene (Accession No: U07050), or an insertion of at least one nucleotide at a position corresponding to position 926/927 of the MRP1 gene (Accession No: U07050);
(d) a polynucleotide capable of hybridizing to a MRP1 gene, wherein said polynucleotide is having a C at a position corresponding to position 95 of the MRP1 gene (Accession No: AF022831), 124667 of the MRP1 gene (Accession No: AC026452), a G at a position corresponding to position 249 of the MRP1 gene (Accession No: AF022830), 259 of the MRP1 gene (Accession No: AF022831), 381 of the MRP1 gene (Accession No: U07050), or an insertion of a T at a position corresponding to position 926/927 of the MRP1 gene (Accession No: U07050);
(e) a polynucleotide encoding an MRP1 polypeptide or fragment thereof, wherein said polypeptide comprises an amino acid substitution at a position corresponding to position 329 of the MRP1 polypeptide (Accession No: G2828206); and
(f) a polynucleotide encoding an MRP1 polypeptide or fragment thereof, wherein said polypeptide comprises an amino acid substitution of Phe to Cys at a position corresponding to position 329 of the MRP1 polypeptide (Accession No: G2828206).

The explanations and interpretations of the terms made above can be applied mutatis mutandis.

The present invention also relates to a method of treating colorectal cancer, cervical cancer, gastric cancer, lung cancer, malignant glioma, ovarian cancer, and pancreatic cancer comprising:
(a) determining the presence or absence of a variant allele comprising a polynucleotide referred to herein; and
(b) administering to a subject a therapeutically effective dosage of irinotecan.

The definitions used in accordance with the use of the present invention apply mutatis mutandis to the above method. Further, all embodiments described in accordance with the use of the present invention can be applied mutatis mutandis to the method of the present invention. Moreover, also encompassed by the method of the present invention are any further developments of said method which the person skilled in the art can make without undue burden based on its knowledge and the prior art, such as those documents referred to throughout this specification.

In a preferred embodiment of the use of the present invention a nucleotide deletion, addition and/or substitution comprised by said polynucleotide results in an altered expression of the variant allele compared to the corresponding wild type allele.

As discussed above, the alleles referred to in accordance with the use of the present invention correspond to the MRP1 gene. It is well known in the art that genes comprise structural elements which encode an amino acid sequence as well as regulatory elements which are involved in the regulation of the expression of said genes. Structural elements are represented by exons which may either encode an amino acid sequence or which may code for RNA which is not encoding an amino acid sequence but is nevertheless involved in RNA function, e.g. by regulating the stability of the RNA or the nuclear export of the RNA.

Regulatory elements of a gene may comprise promoter elements or enhancer elements both of which could be involved in transcriptional control of gene expression. It is very well known in the art that a promoter is to be found upstream of the structural elements of a gene. Regulatory elements such as enhancer elements, however, can be found distributed over the entire locus of a gene. Said elements could reside, e.g., in introns, regions of genomic DNA which separate the exons of a gene. Promoter or enhancer elements correspond to polynucleotide fragments which are capable of attracting or binding polypeptides involved in the regulation of the gene comprising said promoter or enhancer elements. For example, polypeptides involved in regulation of said gene comprise the so called transcription factors.

Said introns may comprise further regulatory elements which are required for proper gene expression. Introns are usually transcribed together with the exons of a gene resulting in a nascent RNA transcript which contains both, exon and intron sequences. The intron encoded RNA sequences are usually removed by a process known as RNA splicing. However, said process also requires regulatory sequences present on a RNA transcript said regulatory sequences may be encoded by the introns.

In addition, besides their function in transcriptional control and control of proper RNA processing and/or stability, regulatory elements of a gene could be also involved in the control of genetic stability of a gene locus. Said elements control, e.g., recombination events or serve to maintain a certain structure of the DNA or the arrangement of DNA in a chromosome.

Therefore, single nucleotide polymorphisms can occur in exons of an allele of a gene which encode an amino acid sequence as discussed supra as well as in regulatory regions which are involved in the above discussed process. The polymorphisms comprised by the polynucleotides referred to in accordance with the use of the present invention can influence the expression level of MRP1 protein via mechanisms involving enhanced or reduced transcription of the MRP1 gene, stabilization of the gene's RNA transcripts and alteration of the processing of the primary RNA transcripts.

Methods for the determination of an altered expression of a variant allele when compared to its wild type counterpart are well known in the art and comprise inter alia those referred to herein above, e.g., PCR based techniques, RFLP-based techniques, DNA sequencing-based techniques, hybridization techniques, Single strand conformational polymorphism (SSCP), denaturating gradient gel electrophoresis (DGGE), mismatch cleavage detection, heteroduplex analysis, techniques based on mass spectroscopy, HPLC-based techniques, primer extension-based techniques, and 5′-nuclease assay-based techniques. It might be necessary to obtain a sample comprising biological material, such as isolated cells or tissue from the subject prior to perform said methods for determination of the expression levels of the wild type and the variant alleles, respectively. An altered expression in accordance with the use of the present invention means that the expression of the wild type allele differs significantly from the expression of the variant allele. A significant difference can be determined by standard statistical methods, such as Student's t-test, chi²-test or the U-test according to Mann and Whitney. Moreover, the person skilled in the art can adopt these and other statistical method known in the art individually without an undue burden.

In a more preferred embodiment of the use of the invention said altered expression is decreased or increased expression.

To determine whether the expression of an allele referred to in accordance to the present invention is increased or decreased in comparison to the corresponding wild type allele well known methods such as PCR based techniques, RFLP-based techniques, DNA sequencing-based techniques, hybridization techniques, Single strand conformational polymorphism (SSCP), denaturating gradient gel electrophoresis (DGGE), mismatch cleavage detection, heteroduplex analysis, techniques based on mass spectroscopy, HPLC-based techniques, primer extension-based techniques, and 5′-nuclease assay-based techniques can be applied. As discussed above, it might be necessary to obtain a sample comprising cells or tissue from the subject in order to determine the expression level of the variant allele referred to in the use of the invention. A decrease or increase of the expression is characterized by a significant difference in the expression level of the variant versus the wild type allele in those assays. Also encompassed by decreased expression is the absence detectable expression of a variant allele.

In a furthermore preferred embodiment of the use of the present invention a nucleotide deletion, addition and/or substitution comprised by said polynucleotide results in an altered activity of the polypeptide encoded by the variant allele compared to the polypeptide encoded by the corresponding wild type allele.

As discussed supra, the variant alleles comprising those polynucleotides specified herein which correspond to coding regions of the MRP1 gene effect the amino acid sequences of the polypeptides encoded by said variant alleles. The variant polypeptides, therefore, exhibit altered biological and/or immunological properties when compared to their corresponding wild type counterpart. Preferred variant polypeptides in accordance with the use of the invention are those, which exhibit an altered biological activity, i.e. altered enzymatic function resulting in reduced, enhanced or complete loss of catalytic activity or altered transport function resulting in reduced, enhanced or complete loss of transport activity or altered binding to receptors or other drug targets resulting in altered activation of signal transduction pathways or altered inhibition of transporter or enzyme function. It might be necessary to obtain a sample comprising biological material such as isolated cells or tissue from the subject prior to perform said methods for determination of the activities of the wild type and the variant polypeptides, respectively. Whether a variant polypeptide has an altered activity or level of expression compared to its wild type corresponding counterpart can be determined by standard techniques well known in the art. Such standard techniques may comprise, e.g., ELISA based assays, RIA based assays, HPLC-based assays, mass spectroscopy-based assays, western blot analysis or assays which are known in the art and described in [Keppler, et al., 1997, Biol Chem 378:787-91, Suzuki, et al., 1994, Adv Prostaglandin Thromboxane Leukot Res 22:83-9, Scheffer, et al., 2000, Cancer Res 60:5269-77, Konig, et al., 1999, Biochim Biophys Acta 1461:377-94, Kool, et al., 1997, Cancer Res 57:3537-47, Bakos, et al., 2000, Mol Pharmacol 57:760-8, Keppler, et al., 1998, Chem Biol Interact 112:153-61, Leier, et al., 2000, Kidney Int 57:1636-42, Evers, et al., 2000, Br J Cancer 83:366-74, Evers, et al., 2000, Br J Cancer 83:375-83] for MRP1.

An altered activity in accordance with the use of the present invention means that the activity of the wild type polypeptide differs significantly from the variant polypeptide. A significant difference can be determined by standard statistical methods referred to herein above.

Most preferably, said altered activity is decreased or increased activity. As discussed for the increase or decrease of expression, a decrease or increase of the activities is characterized by a significant difference in the activity of the variant versus the wild type polypeptide in the assays referred to herein. Also encompassed by decreased activity is the absence detectable activity of a variant allele.

Moreover, in a further preferred embodiment of the use of the present invention said subject is an animal.

As described supra, the subject in accordance with the use of the present invention encompasses animals. The term “animal” as used herein encompasses all animals, preferably animals belonging to the vertebrate family, more preferably mammals. Moreover, the animals can be genetically engineered by well known techniques comprising transgenesis and homologous recombination in order to incorporate one or more of the polynucleotides referred to supra into the genome of said animals. Said animals comprising the genetically engineered animals can be used to study the pharmacological effects of drugs or pro-drugs which are based on the substances or derivatives thereof referred to herein, preferably irinotecan.

In accordance with the foregoing, most preferably, said animal is a mouse or rat.

Said animals are particularly well suited for assaying the pharmacological properties of the substances or derivatives referred to in accordance with the use of the present invention as described in detail in Giovanella, et al., 1991, Cancer Res 51:3052-5, Kunimoto, et al., 1987, Cancer Res 47:5944-7, Kaneda, et al., 1990, Cancer Res 50:1715-20.

Preferably, said mouse is lacking functional MRP1. It is well known in the art how said mice lacking functional cytochrome P450, MRP1 or MDR1 can be obtained. For instance said mice might be generated by homologous recombination as described for MRP1 in Rappa, et al., 2000, Biochemistry 39:3304-10, in Schinkel, 1998, Int J Clin Pharmacol Ther 36:9-13, Schinkel, et al., 2000, Pharmacogenetics 10:583-90.

Moreover, in another preferred embodiment of the use of the present invention said subject is a human.

In particular, the present invention is applicable to humans as is evident from the above. The use of the present invention is to be applied in order to treat or prevent side effects in patients which suffer from colorectal cancer, cervical cancer, gastric cancer, lung cancer, malignant glioma, ovarian cancer, and pancreatic cancer. The pharmacological effects of the above substances or derivatives thereof are well described in humans. However, the conventional therapies do not take into account the individual genetic makeup of the patient. Ethnical populations have different genetic backgrounds, which can also influence the function or regulation of a variant allele and thereby alter the pharmacological response of a patient to a substance or derivative used as a basis for a drug or pro-drug in accordance with the invention.

In light of the foregoing, most preferably, said human is African or Asian.

The Asian population (16%) who shows compared to Caucasians (39%) a lower frequency of the UGT1A1 low expresser genotype (homozygously wildtype at positions corresponding to positions 174990 to 174993 of the UGT1A1 gene Acc. No. GI:11118740) and is therefore less likely to suffer from irinotecan toxicity. On the other hand, this allele is more common in Africans (43%) who have additionally another low expressor allele (insertion of TA at positions corresponding to positions 174989/174990 of the UGT1A1 gene Acc. No. GI:11118740) the homozygous genotype of which occurs in 7%. Africans are therefore more susceptible to irinotecan-related adverse events (population frequency data are from [Beutler, et al., 1998, Proc Natl Acad Sci U S A 95:8170-4, Lampe, et al., 1999, Pharmacogenetics 9:341-9, Hall, et al., 1999, Pharmacogenetics 9:591-9]).

The present invention also relates to a method for selecting a suitable therapy for a subject suffering from colorectal cancer, cervical cancer, gastric cancer, lung cancer, malignant glioma, ovarian cancer, and pancreatic cancer, wherein said method comprises:
(a) determining the presence or absence of a variant allele referred to above in the genome of a subject in a sample obtained from said subject; and
(b) selecting a suitable therapy for said subject based on the results obtained in (a).

The definitions and explanations of the terms made above apply mutatis mutandis to the above method.

The term “suitable therapy” as used herein means that a substance according to the invention is selected and said substance being administered in a certain dosage to a subject, wherein said substance and said dosage are selected based on the knowledge of the presence or absence of a variant allele referred to in accordance with the use of the invention. Said substance and said dosage of the substance are selected in a way that on one hand they are most effective in treating colorectal cancer, cervical cancer, gastric cancer, lung cancer, malignant glioma, ovarian cancer, and pancreatic cancer on the other hand they do not cause toxic or undesirable side effects.

As is evident from the above, a prerequisite for selecting a suitable therapy is the knowledge of the presence or absence of a variant allele referred to in accordance with the use of the invention. Therefore, the method of the present invention encompasses the determination of the presence or absence of said variant alleles in a sample which has been obtained from said subject. The sample which is obtained by the subject comprises biological material which is suitable for the determination of the presence or absence of said variant alleles, such as isolated cells or tissue. Methods for the determination of the presence or absence of the variant alleles of the method of the invention comprise those methods referred to herein above.

Thanks to the method of the present invention, it is possible to efficiently select a suitable therapy for a subject, preferably a human, suffering from colorectal cancer, cervical cancer, gastric cancer, lung cancer, malignant glioma, ovarian cancer, and pancreatic cancer Thereby, mistreatment of patients based on wrong medications and the results thereof, such as development of resistance towards cancer therapy, and subsequent increased costs in health care, can be efficiently avoided. Furthermore, patients that are at high risk can be excluded from therapy prior to the first dose and/or dosage can be adjusted according to the individual's genetic makeup prior to the onset of drug therapy. Also, inhibitors for the mentioned transporter genes (e.g. MRP1) can be applied in genetically defined patient subpopulations. Thus, adverse effects can be avoided and the optimal drug level can be reached faster without time-consuming and expensive drug monitoring-based dose finding. This can reduce costs of medical treatment and indirect costs of disease (e.g. shorter time and less frequent hospitalization of patients).

The following 27 items are also encompassed by the present invention. The definitions and explanations made supra apply mutatis mutandis to the terms used to characterize the items.

1. A method of using irinotecan to treat a patient suffering from cancer which comprises:
(1) determining if the patient has one or more variant alleles of the MRP1 gene in the cancerous tissue;
(2) in a patient having one or more of such variant alleles, administering to the patient an amount of irinotecan which is sufficient to treat a patient having such variant alleles which amount is increased or decreased in comparison to the amount that is administered without regard to the patient's alleles in the MRP1 gene.

2. The method of item 1 wherein the cancer is colorectal cancer, cervical cancer, gastric cancer, lung cancer, malignant glioma, ovarian cancer, or pancreatic cancer.

3. The method of item 2 in which:
(1) the one or more variant alleles result in the patient expressing low amounts of the MRP1 gene product, whereby the amount of irinotecan administered to the patient is decreased to avoid toxicity; or
(2) the one or more variant alleles result in the patient expressing high amounts of the MRP1 gene product, whereby the amount of irinotecan administered to the patient is increased to enhance efficacy.

4. The method of item 3 wherein the one or more variant alleles are in the promoter region of the MRP1 gene.

5. The method of item 3 wherein the one or more variant alleles are in the coding region of the MRP1 gene.

6. The method of item 3 wherein the one or more variant alleles are not in either the promoter region or the coding region of the MRP1 gene.

7. The method of item 3 wherein the one or more variant alleles are in both the promoter region and the coding region of the MRP1 gene.

8. The method of item 3 wherein the one or more variant alleles comprises a polynucleotide selected from the group consisting of:
(a) a polynucleotide having the nucleic acid sequence of any one of SEQ ID NOs:169, 170, 173, 174, 177, 178, 181, 182, 185, 186, 189, 190, 193, 194, 197, 198, 201, 202, 205, 206, 209, 210, 213, 214, 217, 218, 221, 222, 225, 226, 229, 230, 233, 234, 237, 238, 241, 242, 245, 246, 249, 250, 253, 254, 257, 258, 261, 262, 265, 266, 269, 270, 273, 274, 277, 278, 281, 282, 285, 286, 289, 290, 293, 294, 297, 298, 301, 302, 305, 306, 309, 310, 313, 314, 317, 318, 321, 322, 325, 326, 329, 330, 333 and/or 334;
(b) a polynucleotide encoding a polypeptide having the amino acid sequence of any one of SEQ ID NOs: 600, 602 and/or 604;
(c) a polynucleotide capable of hybridizing to a Multidrug Resistance Protein 1 (MRP1) gene, wherein said polynucleotide is having at a position corresponding to positions 57998, 57853, 53282, and/or 39508 of the MRP1 gene (Accession No: GI:7209451), a substitution or deletion of at least one nucleotide or at a position corresponding to positions 137667, 137647, 137710, 124667, and/or 38646 of the MRP1 gene (Accession No: AC026452), a substitution or deletion of at least one nucleotide or at a position corresponding to positions 27258, 27159, 34218, 34215, 55472, and/or 34206 to 34207 of the MRP1 gene (Accession No: AC003026), a substitution or deletion of at least one nucleotide or at a position corresponding to positions 21133, 14008, 18067, 17970, 17900, and/or 18195 of the MRP1 gene (Accession No: U91318), a substitution or deletion of at least one nucleotide or at a position corresponding to positions 79, 88, and/or 249 of the MRP1 gene (Accession No: AF022830), a substitution or deletion of at least one nucleotide or at a position corresponding to positions 95 and/or 259 of the MRP1 gene (Accession No: AF022831), a substitution or deletion of at least one nucleotide or at a position corresponding to positions 150727 and/or 33551 of the MRP1 gene (Accession No: AC025277), a substitution or deletion of at least one nucleotide or at a position corresponding to position 174 of the MRP1 gene (Accession No: AF022828), a substitution or deletion of at least one nucleotide or at a position corresponding to positions 248 and/or 258 of the MRP1 gene (Accession No: AF022829), a substitution or deletion of at least one nucleotide or at a position corresponding to positions 1884, 1625, 1163, 381, 233, 189, 440, and/or 1720 to 1723 of the MRP1 gene (Accession No: U07050), a substitution or deletion of at least one nucleotide or at a position corresponding to positions 926927 and/or 437/438 of the MRP1 gene (Accession No: U07050) a insertion of at least one nucleotide or at a position corresponding to position 55156/55157 of the MRP1 gene (Accession No: AC003026) a insertion of at least one nucleotide;
(d) a polynucleotide capable of hybridizing to a MRP1 gene, wherein said polynucleotide is having at a position corresponding to position 21133, 14008 and/or 18195 of the MRP1 gene (Accession No: U91318) or at a position corresponding to position 27258 and/or 34218 of the MRP1 gene (Accession No: AC003026) or at a position corresponding to position 79 of the MRP1 gene (Accession No: AF022830) or at a position corresponding to position 57998, and/or 57853 of the MRP1 gene (Accession No: GI:7209451) or at a position corresponding to position 137667 and/or 137647 of the MRP1 gene (Accession No: AC026452) or at a position corresponding to position 150727 and/or 33551 of the MRP1 gene (Accession No: AC025277) or at a position corresponding to position 248 of the MRP1 gene (Accession No: AF022829) or at a position corresponding to position 1884, 1625, 233, and/or 189 of the MRP1 gene (Accession No: U07050) an A, at a position corresponding to position 39508 of the MRP1 gene (Accession No: GI:7209451) or at a position corresponding to position 17900, 18067 and/or 18195 of the MRP1 gene (Accession No: U91318) or at a position corresponding to position 174 of the MRP1 gene (Accession No: AF022828) or at a position corresponding to position 440 and/or 1163 of the MRP1 gene (Accession No: U07050) a T, at a position corresponding to position 88 of the MRP1 gene. (Accession No: AF022830) or at a position corresponding to position 95 of the MRP1 gene (Accession No: AF022831) or at a position corresponding to position 27159, 55472 and/or 34215 of the MRP1 gene (Accession No: AC003026) or at a position corresponding to position 124667 and/or 38646 of the MRP1 gene (Accession No: AC026452) or at a position corresponding to position 53282 of the MRP1 gene (Accession No: GI:7209451) or at a position corresponding to position 137710 of the MRP1 gene (Accession No: AC026452) a C, at a position corresponding to position 249 of the MRP1 gene (Accession No: AF022830) or at a position corresponding to position 258 of the MRP1 gene (Accession No: AF022829) or at a position corresponding to position 259 of the MRP1 gene (Accession No: AF022831) or at a position corresponding to position 381 of the MRP1 gene (Accession No: U07050) a G, at a position corresponding to position 17970 of the MRP1 gene (Accession No: U91318) a deletion of a T or at a position corresponding to position 34206 to 34207 of the MRP1 gene (Accession No: AC003026) a deletion of a AT or at a position corresponding to position 1720 to 1723 of the MRP1 gene (Accession No: U07050) a deletion of GGTA, at a position corresponding to position 926/927 a insertion of a T and/or 437/438 of the MRP1 gene (Accession No: U07050) a insertion of a TCCTTCC, at a position corresponding to position 55156/55157 of the MRP1 gene (Accession No: AC003026) a insertion of TGGGGC;
(e) a polynucleotide encoding an MRP1 polypeptide or fragment thereof, wherein said polypeptide comprises an amino acid substitution at a position corresponding to positions 600, 602, and/or 604 of the MRP1 polypeptide (Accession No: G2828206);
(f) a polynucleotide encoding an MRP1 polypeptide or fragment thereof, wherein said polypeptide comprises an amino acid substitution of Phe to Cys at a position corresponding to position 239 of the MRP1 polypeptide (Accession No: G2828206) or/and Arg to Ser at a position corresponding to position 433 of the MRP1 polypeptide (Accession No: G2828206) or/and Arg to Gin at a position corresponding to position 723 of the MRP1 polypeptide (Accession No: G2828206);

9. The method of item 8 wherein the one or more variant alleles comprises a polynucleotide selected from the group consisting of:
(a) a polynucleotide having the nucleic acid sequence of any one of SEQ ID NO: 181, 209, 217, 205, 277, 281, 301, 325, 229, 193, 313, 293 or 253:
(b) a polynucleotid encoding a polypeptide having the amino acid sequence of SEQ ID NO: 600;
(c) a polynucleotide capable of hybridizing to a MRP1 gene, wherein said polynucleotide is having a substitution at a position corresponding to position 137647 of the MRP1 gene (Accession No: AC026452), 95 of the MRP1 gene (Accession No: AF022831), 53282 of the MRP1 gene (Accession No: GI:7209451), 249 of the MRP1 gene (Accession No: AF022830), 259 of the MRP1 gene (Accession No: AF022831),124667 of the MRP1 gene (Accession No: AC026452), 381, 440, 1625 of the MRP1 gene (Accession No: U07050), 34218 of the MRP1 gene (Accession No: AC003026), 18067 or 17900 of the MRP1 gene (Accession No: U91318) or an insertion of at least one nucleotide at a position corresponding to position 926/927 of the MRP1 gene (Accession No: U07050);
(d) a polynucleotide capable of hybridizing to a MRP1 gene, wherein said polynucleotide is having a T at a position corresponding to position 137647 of the MRP1 gene (Accession No: AC026452), 18067 or 17900 of the MRP1 gene (Accession No: U91318), 440 of the MRP1 gene (Accession No: U07050), a C at a position corresponding toposition 95 of the MRP1 gene (Accession No: AF022831), 124667 of the MRP1 gene (Accession No: AC026452), a G at a position corresponding to position 53282 of the MRP1 gene (Accession No: GI:7209451), 249 of the MRP1 gene (Accession No: AF022830), 259 of the MRP1 gene (Accession No: AF022831), 381 of the MRP1 gene (Accession No: U07050), or an A at a position corresponding to position 34218 of the MRP1 gene (Accession No: AC003026) or 1625 of the MRP1 gene (Accession No: U07050) or an insertion of a T at a position corresponding to position 926/927 of the MRP1 gene (Accession No: U07050);
(e) a polynucleotide encoding an MRP1 polypeptide or fragment thereof, wherein said polypeptide comprises an amino acid substitution at a position corresponding to position 329 of the MRP1 polypeptide (Accession No: G2828206); and
(f) a polynucleotide encoding an MRP1 polypeptide or fragment thereof, wherein said polypeptide comprises an amino acid substitution of Phe to Cys at a position corresponding to position 329 of the MRP1 polypeptide (Accession No: G2828206).

10. The method of item 8 in which the one or more variant alleles results in the patient expressing low amounts of the MRP1 gene product, whereby the amount of irinotecan administered to the patient is decreased.

11. The method of item 8 in which the one or more variant alleles results in the patient expressing high amounts of the MRP1 gene product, whereby the amount of irinotecan administered to the patient is increased.

12. The method of item 9 in which the one or more variant alleles results in the patient expressing low amounts of the MRP1 gene product, whereby the amount of irinotecan administered to the patient is decreased.

13. The method of item 9 in which the one or more variant alleles results in the patient expressing high amounts of the MRP1 gene product, whereby the amount of irinotecan administered to the patient is increased.

14. A method for determining whether a patient is at risk for a toxic reaction to treatment with irinotecan which comprises determining if the patient has one or more variant alleles of the MRP1 gene.

15. The method of item 14 which further comprises administering to the patient reduced amounts of irinotecan.

16. A method for determining the optimum treatment regimen for administering irinotecan to a patient suffering from cancer which comprises:
(1) determining if the patient has one or more variant alleles of the MRP1 gene;
(2) in a patient having one or more of such alleles increasing or decreasing the amount of irinotecan in comparison to the amount that is administered without regard to the patient's alleles in the MRP1 gene.

17. A method of treating cancer in a patient having one or more variant alleles of the MRP1 gene such that expression levels of the MRP1 gene product are lower than in the general population and so indicates high sensitivity to irinotecan which comprises administering to the patient a decreased amount of irinotecan.

18. A method of treating cancer in a patient having one or more variant alleles of the MRP1 gene such that expression levels of the MRP1 gene product are higher than in the and so indicates resistance or predisposition to resistance to irinotecan which comprises administering to the patient an increased amount of irinotecan.

19. The method of item 18 in which patients that have a variant allele that indicates resistance or predisposition to resistance are treated with an MRP1 inhibitor.

20. The method of item 19 wherein the MRP1 inhibitor is selected from the group consisting of: SDZ-PSC 833, SDZ 280-446, MK571, MS209 (quinolone derivative), PAK-104p, Verapamil, Benzbromarone, Dipyridamole, Furosemide, Gamma-GS(naphtyl)cysteinyl-glycine diethyl ester, Genistein, Quinidine, Rifampicin, RU 486, Sulfinpyrazone.

21. The method of item 17 which further comprises monitoring the patient during treatment by assaying for changes in expression levels of the MRP1 gene product in the cancerous cells whereby an increase in the expression level of the MRP1 gene product is compensated for by an increase in the amount of irinotecan administered to the patient.

22. A method of treating cancer in a patient which comprises internally administering to the patient an effective amount of irinotecan, wherein the treatment regimen is modified based upon the genotype of the patient's MRP1 gene.

23. A method of treating a population of patients suffering from cancer which comprises:
(1) determining, on a patient by patient basis, if the patient has one or more variant alleles of the MRP1 gene;
(2) in a patient having one or more of such variant alleles, administering to the patient an amount of irinotecan which is sufficient to treat a patient having such variant alleles which amount is increased or decreased in comparison to the amount that is administered without regard to the patient's alleles in the MRP1 gene.

24. A method for predicting sensitivity to irinotecan in a patient suffering from cancer which comprises determining if the patient has one or more variant alleles of the MRP1 gene, which alleles indicate that the cancerous cells express low or high amounts of the MRP1 gene product, whereby low expression indicates high sensitivity to irinotecan and high expression indicates resistance or predisposition to resistance to irinotecan.

25. The method of item 24 in which patients that have a genotype that indicates resistance or predisposition to resistance are treated with a MRP1 inhibitor.

26. The method of item 25 wherein the MRP1 inhibitor is selected from the group consisting of: SDZ-PSC 833, SDZ 280-446, MK571, MS209 (quinolone derivative), PAK-104p, Verapamil, Benzbromarone, Dipyridamole, Furosemide, Gamma-GS(naphtyl)cysteinyl-glycine diethyl ester, Genistein, Quinidine, Rifampicin, RU 486, Sulfinpyrazone.

27. The method of item 26 wherein the patients that have a genotype that indicates resistance or predisposition to resistance are monitored during treatment by assaying for expression levels of the MRP1 gene product in the cancerous cells.

The decreased expression as referred to herein above includes in addition to a significantly decreased amount of transcripts encoding a functional gene product also a normal or even elevated amount of transcripts encoding a gene product which has no activity or a significantly decreased activity.

By “in comparison to the amount that is administered without regard to the patient's alleles in the MDR1 gene” a standard dose is meant which is routinely administered to patients in need thereof without regarding the genotype. Such a general population of patients is considered as having the normal genotype, i.e. wildtype genotype.

Further, the present invention encompasses a method for improving and/or modifying a therapy comprising determining the expression levels of MRP1, hereinafter referred to as expression profile or the protein level of the MRP1 protein, hereinafter referred to as the protein profile, or the activity level of the said protein, hereinafter referred to as the activity profile.

The term “expression level” as referred to in the context of the present invention means the detectable amount of transcripts of the MRP1 gene relative to the amount of transcripts for a housekeeping gene, such as PLA2. The amount of transcripts can be determined by standard molecular biology techniques including Northern analysis, RNAse protection assays, PCR based techniques encompassing Taq-Man analysis. Preferably, the determination can be carried out as described in the accompanied Examples 4 and 5. The term “expression profile” means that the expression level of a panel of the aforementioned genes is determined and the expression levels are compared to a reference standard. As a reference standard, preferably transcripts are obtained from cells or tissues of a subject having the aforementioned wildtype alleles of the respective genes in their genomes.

The term “protein level” refers to the detectable amount of MRP1 relative to the amount of a protein encoded by a housekeeping gene, such as PLA2. The amount of proteins can be determined by standard biochemical techniques, such as Western analysis, ELISA, RIA or other antibody based techniques known in the art. The term “protein profile” means that the protein level of a panel of the aforementioned proteins is determined and the protein levels are compared to a reference standard. As a reference standard, preferably proteins are obtained from cells or tissues of a subject having the aforementioned wildtype alleles of the respective genes in their genomes.

The term “activity level” means the detectable biological activity of MRP1 relative to the activity or amount of a encoded by the allellic variants of the gene as disclosed in the present invention relative to the activity of the protein encoded by the corresponding wild-type allele of the gene. Biological assays for the aforementioned proteins are well known in the art and described in Hallo et al., Anticancer Res. 1998, 18:2981-7. As a reference standard, preferably proteins are obtained from cells or tissues of a subject having the aforementioned wildtype alleles of the respective genes in their genomes.

The aforementioned methods, preferably, comprise the steps (i) obtaining a tumor sample from a patient during specific stages of a tumor therapy; and (ii) determining the expression profile, protein profile or activity profile for MRP1. Based on the expression profiles a clinician can efficiently adapt the therapy. This comprises inter alia dosage adjustment and/or including administration of an MRP1 inhibitor. Preferably, said inhibitor is selected from the following group of inhibitors: SDZ-PSC 833, SDZ 280-446, MK571, MS209 (quinolone derivative), PAK-104p, Verapamil, Benzbromarone, Dipyridamole, Furosemide, Gamma-GS(naphtyl)cysteinyl-glycine diethyl ester, Genistein, Quinidine, Rifampicin, RU 486, Sulfinpyrazone, tricyclic isoxazole (e.g. LY 402913) (http://bigfoot.med.unc.edu/watkinsLab/intesinfo.htm, Paul Watkins, University of North Carolina).

Finally, the present invention encompasses a method for determining whether a patient has developed a resistance against the drugs referred to in the context of the present invention. Said method comprising the steps of (i) obtaining a tumor sample from a patient during specific stages of a tumor therapy; and (ii) determining the expression level of MRP1. The expression of said gene can be determined as described in Examples 4 and 5 or as described above. Based on the evaluation of said expression profile, a clinician can more efficiently adapt the therapy. This comprises inter alia dosage adjustment and/or including administration of an MRP1 inhibitor as defined supra.

Each of the documents cited herein (including any manufacturer's specifications, instructions, etc.) are hereby incorporated by reference.

The nucleic acid and amino acid sequences referred to in this application by sequence identification numbers (SEQ ID NOs.) are listed in the following Tables 1 2, 3 and 4. For positions of polymorphic nucleotides, the following substitute letters are used in the nucleic acid sequences: R, G or A; Y, T or C; M, A or C; K, G or T; S, G or C; W, A or T.

Amino acid sequences are shown in the one letter code. The letter X at polymorphic amino acid positions represents the modified amino acid or its corresponding wild type amino acid (see accession numbers).

Moreover, all nucleic acid and amino acid sequences referred to herein by making reference to GenBank accession numbers are shown in FIGS. 4 to 29 below

TABLE 1

The nucleic acid and amino acid sequences referred to in this application

| | | | | | |
|---|---|---|---|---|---|
| SEQ | SEQ | SEQ | Sequence | SEQ | Sequence |

| | | | | | | | | | |
|---|---|---|---|---|---|---|---|---|---|
| Vari- | Acc. | ID | Sequence | ID | Sequence | ID | wt > mut | ID | wt > mut |

| | | | | | | | | | | | |
|---|---|---|---|---|---|---|---|---|---|---|---|
| Gene | ation | SNP | no. | NO | forward | NO | reverse | NO | forward | NO | reverse |

| | | | | | | | | | | | |
|---|---|---|---|---|---|---|---|---|---|---|---|
| UGT1A1 | T > G | 59 | GI:8850235 | 001 | GTCCTGGGCCG | 002 | ACACAGCAGCC | 003 | GTCCTGGGCCK | 004 | ACACAGCAGCM |
| | | | | | GCTGCTGTGT | | GGCCCAGGAC | | GCTGCTGTGT | | GGCCCAGGAC |
| UGT1A1 | C > T | 160 | GI:8850235 | 005 | GGCCATCCAGT | 006 | TGCTGCAGCTA | 007 | GGCCATCCAGY | 008 | TGCTGCAGCTR |
| | | | | | AGCTGCAGCA | | CTGGATGGCC | | AGCTGCAGCA | | AGCTGCAGCA |
| UGT1A1 | G > A | 226 | GI:8850235 | 009 | CATCAGAGACA | 010 | TAAAATGCTCTG | 011 | CATCAGAGACR | 012 | TAAAATGCTCYG |
| | | | | | GAGCATTTTA | | TCTCTGATG | | GAGCATTTTA | | TCTCTGATG |
| UGT1A1 | T > A | 539 | GI:8850235 | 013 | TTGCATGCACA | 014 | GCTGCATGGCT | 015 | TTGCATGCACW | 016 | GCTGCATGGCK |
| | | | | | GCCATGCAGC | | GTGCATGCAA | | GCCATGCAGC | | GTGCATGCAA |
| UGT1A1 | T > C | 544 | GI:8850235 | 017 | TGCACTGCCAC | 018 | TCCAGGCTGCG | 019 | TGCACTGCCAY | 020 | TCCAGGCTGCR |
| | | | | | GCAGCCTGGA | | GTGCATGCAA | | GCAGCCTGGA | | GTGCATGCAA |
| UGT1A1 | C > T | 640 | GI:8850235 | 021 | CTTCCTGCAGT | 022 | TTCTTCACCCAC | 023 | CTTCCTGCAGY | 024 | TTCTTCACCCRC |
| | | | | | GGGTGAAGAA | | TGCAGGAAG | | GGGTGAAGAA | | TGCAGGAAG |
| UGT1A1 | C > A | 701 | GI:8850235 | 025 | GTTTATTCCCAG | 026 | GGTTGCATACT | 027 | GTTTATTCCCM | 028 | GGTTGCATACK |
| | | | | | TATGCAACC | | GGGAATAAAC | | GTATGCAACC | | GGGAATAAAC |
| UGT1A1 | G > C | 841 | GI:8850235 | 029 | GGTTTTTGTTCG | 030 | TTGATTCCACGA | 031 | GGTTTTTGTTSG | 032 | TTGATTCCACSA |
| | | | | | TGGAATCAA | | ACAAAAACC | | TGGAATCAA | | ACAAAAACC |
| UGT1A1 | C > A | 855 | GI:8850235 | 033 | GAATCAACTGA | 034 | TTTGGTGAAGT | 035 | GAATCAACTGM | 036 | TTTGGTGAAGK |
| | | | | | CTTCACCAAA | | CAGTTGATTC | | CTTCACCAAA | | CAGTTGATTC |
| UGT1A1 | C > T | 890 | GI:8850235 | 037 | GAATTTGAAGTC | 038 | ATTAATGTAGAC | 039 | GAATTTGAAGY | 040 | ATTAATGTAGRC |
| | | | | | TACATTAAT | | TTCAAATTC | | CTACATTAAT | | TTCAAATTC |
| UGT1A1 | G > A | 938 | GI:8850235 | 041 | TTCTCTTTGGAA | 042 | GACCATTGATTC | 043 | TTCTCTTTGGRA | 044 | GACCATTGATY |
| | | | | | TCAATGGTC | | CAAAGAGAA | | TCAATGGTC | | CCAAAGAGAA |
| UGT1A1 | C > T | 1006 | GI:8850235 | 045 | CAAAATCCCTTA | 046 | AGGACTGTCTA | 047 | CAAAATCCCTYA | 048 | AGGACTGTCTR |
| | | | | | GACAGTCCT | | AGGGATTTTG | | GACAGTCCT | | AGGGATTTTG |
| UGT1A1 | A > G | 1007 | GI:8850235 | 049 | AAAATCCCTCG | 050 | CAGGACTGTCC | 051 | AAAATCCCTCR | 052 | CAGGACTGTCY |
| | | | | | GACAGTCCTG | | GAGGGATTTT | | GACAGTCCTG | | GAGGGATTTT |
| UGT1A1 | G > A | 1020 | GI:8850235 | 053 | CAGTCCTGTGA | 054 | CAGTGTACCGT | 055 | CAGTCCTGTGR | 056 | CAGTGTACCGY |
| | | | | | CGGTACACTG | | CACAGGACTG | | CGGTACACTG | | CACAGGACTG |
| UGT1A1 | C > T | 1084 | GI:8850235 | 057 | GTGGCTACCCT | 058 | AGATCGTTTTAG | 059 | GTGGCTACCCY | 060 | AGATCGTTTTRG |
| | | | | | AAAACGATCT | | GGTAGCCAC | | AAAACGATCT | | GGTAGCCAC |
| UGT1A1 | A > G | 1085 | GI:8850235 | 061 | TGGCTACCCCG | 062 | CAGATCGTTTC | 063 | TGGCTACCCCR | 064 | CAGATCGTTTY |
| | | | | | AAACGATCTG | | GGGGTAGCCA | | AAACGATCTG | | GGGGTAGCCA |
| UGT1A1 | C > G | 1114 | GI:8850235 | 065 | CCCGATGACCG | 066 | ATAAAGGCACC | 067 | CCCGATGACCS | 068 | ATAAAGGCACS |
| | | | | | GTGCCTTTAT | | GGTCATCGGG | | GTGCCTTTAT | | GGTCATCGGG |
| UGT1A1 | G > A | 1117 | GI:8850235 | 069 | GATGACCCGTA | 070 | GTGATAAAGGT | 071 | GATGACCCGTR | 072 | GTGATAAAGGY |
| | | | | | CCTTTATCAC | | ACGGGTCATC | | CCTTTATCAC | | ACGGGTCATC |
| UGT1A1 | C > T | 1139 | GI:8850235 | 073 | CATGCTGGTTT | 074 | AACACCATGGA | 075 | CATGCTGGTTY | 076 | AACACCATGGR |
| | | | | | CCATGGTGTT | | AACCAGCATG | | CCATGGTGTT | | AACCAGCATG |
| UGT1A1 | C > G | 1158 | GI:8850235 | 077 | TTTATGAAAGGA | 078 | CATTGCATATCC | 079 | TTTATGAAAGSA | 080 | CATTGCATATSC |
| | | | | | TATGCAATG | | TTTCATAAA | | TATGCAATG | | TTTCATAAA |
| UGT1A1 | CC > | 1175 to | GI:8850235 | 081 | AATGGCGTTCG | 082 | TCATCACCATAC | 083 | AATGGCGTTCY | 084 | TCATCACCATSR |
| | GT | 1176 | | | TATGGTGATGA | | GAACGCCATT | | ATGGTGATGA | | GAACGCCATT |
| UGT1A1 | G > C | 1216 | GI:8850235 | 085 | GATGGACAATC | 086 | ATGCGCTTTGG | 087 | GATGGACAATS | 088 | ATGCGCTTTGS |
| | | | | | CAAAGCGCAT | | ATTGTCCATC | | CAAAGCGCAT | | ATTGTCCATC |
| UGT1A1 | A > G | 1297 | GI:8850235 | 089 | AAATGCTCTAGA | 090 | ATGACTGCTTCT | 091 | AAATGCTCTARA | 092 | ATGACTGCTTYT |
| | | | | | AGCAGTCAT | | AGAGCATTT | | AGCAGTCAT | | AGAGCATTT |
| UGT1A1 | A > T | 1324 | GI:8850235 | 093 | CAAAAGTTACTA | 094 | ATGTTCTCCTAG | 095 | CAAAAGTTACW | 096 | ATGTTCTCCTW |
| | | | | | GGAGAACAT | | TAACTTTTG | | AGGAGAACAT | | GTAACTTTTG |
| UGT1A1 | T > G | 1471 | GI:8850235 | 097 | CTGGTACCAGG | 098 | AAGGAATGGTC | 099 | CTGGTACCAGK | 100 | AAGGAATGGTM |
| | | | | | ACCATTCCTT | | CTGGTACCAG | | ACCATTCCTT | | CTGGTACCAG |
| UGT1A1 | C > T | 1478 | GI:8850235 | 101 | CAGTACCATTTC | 102 | CACGTCCAAGA | 103 | CAGTACCATTYC | 104 | CACGTCCAAGR |
| | | | | | TTGGACGTG | | AATGGTACTG | | TTGGACGTG | | AATGGTACTG |
| UGT1A1 | de/CT | 372 to | GI:8850235 | 105 | TAAAAAAGGAC | 106 | AGCATAGCAGT | 107 | TAAAAAAGGAnC | 108 | AGCATAGCAGn |
| | | 373 | | | TGCTATGCT | | CCTTTTTTA | | TGCTATGCT | | TCCTTTTTTA |
| UGT1A1 | de/TT | 523 to | GI:8850235 | 109 | GCCCACTGTAT | 110 | CATGCAAGAAT | 111 | GCCCACTGTAn | 112 | CATGCAAGAAnT |
| | C | 525 | | | TCTTGCATG | | ACAGTGGGC | | TTCTTGCATG | | ACAGTGGGC |
| UGT1A1 | del | 892 to | GI:8850235 | 113 | ATTTGAAGCCT | 114 | ATGTTCTCCAG | 115 | ATTTGAAGCCnT | 116 | ATGTTCTCCAnG |
| | TACA | 905 | | | GGAGAACAT | | GCTTCAAAT | | GGAGAACAT | | GCTTCAAAT |
| | TTA | | | | | | | | | | |
| | ATGC | | | | | | | | | | |
| | TTC | | | | | | | | | | |
| UGT1A1 | insT | 470/ | GI:8850235 | 129 | CTGACGGACCC | 130 | AAGGAAGGAAA | 131 | CTGACGGACCC | 132 | AAGGAAGGAAA |
| | | 471 | | | TTTTCCTTCCTT | | TGGGTCCGTCAG | | nTTTCCTTCCTT | | nGGGTCCGTCAG |
| UGT1A1 | insG | 1222/ | GI:8850235 | 133 | CAATGCAAAGC | 134 | AGTCTCCATGC | 135 | CAATGCAAAGC | 136 | AGTCTCCATGC |
| | | 1223 | | | GGCATGGAGACT | | CGCTTTGCATTG | | nGCATGGAGACT | | nGCTTTGCATTG |
| Cyp3A5 | T > C | 47518 | GI:10281451 | 137 | AAGGACTTCTA | 138 | TAGAAGTCCTT | 139 | AAGGAYTTCTA | 140 | TAGAARTCCTT |
| Cyp3A5 | T > G | 145601 | GI:11177452 | 141 | TGGGCGTGCAA | 142 | TTGCACGCCCA | 143 | TGGGCKTGCAA | 144 | TTGCAMGCCCA |
| Cyp3A5 | A > G | 145929 | GI:11177452 | 145 | GCCCCGCCTCC | 146 | GGAGGCGGGGC | 147 | GCCCCRCCTCC | 148 | GGAGGYGGGGC |
| Cyp3A5 | A > G | 9736 | GI:10281451 | 149 | CTCACGCTGGG | 150 | CCCAGCGTGAG | 151 | CTCACRCTGGG | 152 | CCCAGYGTCTC |
| MRP1 | G > A | 21133 | U91318 | 169 | CCCAAAACACA | 170 | GCAGGGTGTGT | 171 | CCCAAAACACR | 172 | GCAGGGTGTGY |
| | | | | | CACACCCTGC | | GTGTTTTGGG | | CACACCCTGC | | GTGTTTTGGG |
| MRP1 | G > T | 57998 | GI:7209451 | 173 | ACGCTCAGAGT | 174 | AGTCCATGAAA | 175 | ACGCTCAGAGK | 176 | AGTCCATGAAM |
| | | | | | TTCATGGACT | | CTCTGAGCGT | | TTCATGGACT | | CTCTGAGCGT |
| MRP1 | C > T | 137667 | AC026452 | 177 | GCAGGTGGCCT | 178 | AATGTGCACAA | 179 | GCAGGTGGCCY | 180 | AATGTGCACAR |
| | | | | | TGTGCACATT | | GGCCACCTGC | | TGTGCACATT | | GGCCACCTGC |
| MRP1 | C > T | 137647 | AC026452 | 181 | TTGCCGTCTAT | 182 | CAATGGTCACA | 183 | TTGCCGTCTAY | 184 | CAATGGTCACR |
| | | | | | GTGACCATTG | | TAGACGGCM | | GTGACCATTG | | TAGACGGCAA |
| MRP1 | G > A | 27258 | AC003026 | 185 | GATTCTCTCCAA | 186 | GATGTTTTCTTTG | 187 | GATTCTCTCCRA | 188 | GATGTTTTCTYG |
| | | | | | GAAAACATC | | GAGAGAATC | | GAAAACATC | | GAGAGAATC |
| MRP1 | G > A | 14008 | U91318 | 189 | CTGGGAAGTCA | 190 | GGGTCAGGGAT | 191 | CTGGGAAGTCR | 192 | GGGTCAGGGAY |
| | | | | | TCCCTGACCC | | GACTTCCCAG | | TCCCTGACCC | | GACTTCCCAG |
| MRP1 | C > T | 18067 | U91318 | 193 | CCACGGCAGCT | 194 | CCAGGTCCACA | 195 | CCACGGCAGCY | 196 | CCAGGTCCACR |
| | | | | | GTGGACCTGG | | GCTGCCGTGG | | GTGGACCTGG | | GCTGCCGTGG |
| MRP1 | G > A | 79 | AF022830 | 197 | CCAGGCAGCCA | 198 | CAACCTTCAC | 199 | CCAGGCAGCCR | 200 | CAACCTTCACY |
| | | | | | GTGAAGGTTG | | GGCTGCCTGG | | GTGAAGGTTG | | GGCTGCCTGG |
| MRP1 | T > C | 88 | AF022830 | 201 | CGGTGAAGGTC | 202 | AGGAGTACACG | 203 | CGGTGAAGGTY | 204 | AGGAGTACACR |
| | | | | | GTGTACTCCT | | ACCTTCACCG | | GTGTACTCCT | | ACCTTCACCG |
| MRP1 | T > G | 249 | AF022830 | 205 | CTCATGAGCTG | 206 | CTTGAAGAAGC | 207 | CTCATGAGCTK | 208 | CTTGAAGAAGM |
| | | | | | CTTCTTCAAG | | AGCTCATGAG | | CTTCTTCAAG | | AGCTCATGAG |
| MRP1 | T > C | 95 | AF022831 | 209 | AGTTCGTGAAC | 210 | CCTTCGTGTCG | 211 | AGTTCGTGAAY | 212 | CCTTCGTGTCR |
| | | | | | GACACGAAGG | | TTCACGAACT | | GACACGAAGG | | TTCACGAACT |
| MRP1 | C > T | 57853 | GI:7209451 | 213 | GGCAGTGGGCT | 214 | CCACTCCCTCA | 215 | GGCAGTGGGCY | 216 | CCACTCCCTCR |
| | | | | | GAGGGAGTGG | | GCCCACTGCC | | GAGGGAGTGG | | GCCCACTGCC |
| MRP1 | C > G | 53282 | GI:7209451 | 217 | GCCAGTTGGAG | 218 | CCCCAAGTGAC | 219 | GCCAGTTGGAS | 220 | CCCCAAGTGAS |
| | | | | | TCACTTGGGG | | TCCAACTGGC | | TCACTTGGGG | | TCCAACTGGC |
| MRP1 | A > G | 137710 | AC026452 | 221 | ACTCTCACTCG | 222 | TGCTGTGCCCC | 223 | ACTCTCACTCR | 224 | TGCTGTGCCCY |
| | | | | | GGGCACAGCA | | GAGTGAGAGT | | GGGCACAGCA | | GAGTGAGAGT |
| MRP1 | G > C | 27159 | AC003026 | 225 | TCGTTGATCACA | 226 | ACAGACAGATG | 227 | TCGTTGATCASA | 228 | ACAGACAGATS |
| | | | | | TCTGTCTGT | | TGATCAACGA | | TCTGTCTGT | | TGATCAACGA |
| MRP1 | G > A | 34218 | AC003026 | 229 | GTGCACTCACA | 230 | CACCCGGCCAT | 231 | GTGCACTCACR | 232 | CACCCGGCCAY |
| | | | | | TGGCCGGGTG | | GTGAGTGCAC | | TGGCCGGGTG | | GTGAGTGCAC |
| MRP1 | G > C | 34215 | AC003026 | 233 | CATGTGCACTC | 234 | CCGGCCACGTG | 235 | CATGTGCACTS | 236 | CCGGCCACGTS |
| | | | | | ACGTGGCCGG | | AGTGCACATG | | ACGTGGCCGG | | AGTGCACATG |
| MRP1 | G > A | 39508 | GI:7209451 | 237 | GTTTCGTTGTA | 238 | TCCCACCCCCT | 239 | GTTTCGTTGTR | 240 | TCCCACCCCCY |
| | | | | | GGGGGTGGGA | | ACAACGAAAC | | GGGGGTGGGA | | ACAACGAAAC |
| MRP1 | T > C | 55472 | AC003026 | 241 | TGTCTAATTACA | 242 | ATCCATTTCTGT | 243 | TGTCTAATTAYA | 244 | ATCCATTTCTRT |
| | | | | | GAAATGGAT | | AATTAGACA | | GAAATGGAT | | AATTAGACA |
| MRP1 | G > A | 150727 | AC025277 | 245 | CCATGTCAGCA | 246 | ACCTGTGTCAT | 247 | CCATGTCAGCR | 248 | ACCTGTGTCAY |
| | | | | | TGACACAGGT | | GCTGACATGG | | TGACACAGGT | | GCTGACATGG |
| MRP1 | de/T | 17970 | U91318 | 249 | CTGGTTTTTTCT | 250 | TGACCGGAAGA | 251 | CTGGTTTTTTnC | 252 | TGACCGGAAGn |
| | | | | | TCCGGTCA | | AAAAACCAG | | TTCCGGTCA | | AAAAAAACCAG |
| MRP1 | C > T | 17900 | U91318 | 253 | TGTCTCCTTTTG | 254 | TGGGAGAAGCA | 255 | TGTCTCCTTTYG | 256 | TGGGAGAAGCR |
| | | | | | CTTCTCCCA | | AAAGGAGACA | | CTTCTCCCA | | AAAGGAGACA |
| MRP1 | G > A | 18195 | U91318 | 257 | CACTGGCACAA | 258 | CTAGAGGCCAT | 259 | CACTGGCACAR | 260 | CTAGAGGCCAY |
| | | | | | TGGCCTCTAG | | TGTGCCAGTG | | TGGCCTCTAG | | TGTGCCAGTG |
| MRP1 | G > A | 33551 | AC025277 | 261 | TGTGACCACAA | 262 | ACACACTCATTT | 263 | TGTGACCACAR | 264 | ACACACTCATYT |
| | | | | | ATGAGTGTGT | | GTGGTCACA | | ATGAGTGTGT | | GTGGTCACA |
| MRP1 | C > T | 174 | AF022828 | 265 | CCAGGCCCCCT | 266 | CCTGAGGTCTA | 267 | CCAGGCCCCCY | 268 | CCTGAGGTCTR |
| | | | | | AGACCTCAGG | | GGGGGCCTGG | | AGACCTCAGG | | GGGGGCCTGG |
| MRP1 | C > A | 248 | AF022829 | 269 | CCTTTCCACTAC | 270 | GAGGCCACAGT | 271 | CCTTTCCACTM | 272 | GAGGCCACAGK |
| | | | | | TGTGGCCTC | | AGTGGAAAGG | | CTGTGGCCTC | | AGTGGAAAGG |
| MRP1 | C > G | 258 | AF022829 | 273 | CCTGTGGCCTG | 274 | ATCCTGGATTCA | 275 | CCTGTGGCCTS | 276 | ATCCTGGATTSA |
| | | | | | AATCCAGGAT | | GGCCACAGG | | AATCCAGGAT | | GGCCACAGG |
| MRP1 | A > G | 259 | AF022831 | 277 | AAGGTAGGGGG | 278 | TGGCACAGCGC | 279 | AAGGTAGGGGR | 280 | TGGCACAGCGY |
| | | | | | CGCTGTGCCA | | CCCCTACCTT | | CGCTGTGCCA | | CCCCTACCTT |
| MRP1 | T > C | 124667 | AC026452 | 281 | GCGTGCCCAGC | 282 | AAACCCCAGGG | 283 | GCGTGCCCAGY | 284 | AAACCCCAGGR |
| | | | | | CCTGGGGTTT | | CTGGGCACGC | | CCTGGGGTTT | | CTGGGCACGC |
| MRP1 | G > A | 1884 | U07050 | 285 | AGCCTTGGAGA | 286 | CACCCCAGATT | 287 | AGCCTTGGAGR | 288 | CACCCCAGATY |
| | | | | | ATCTGGGGTG | | CTCCAAGGCT | | ATCTGGGGTG | | CTCCAAGGCT |
| MRP1 | G > C | 38646 | AC026452 | 289 | CCTTAAACAGC | 290 | CTTTTCAAATGC | 291 | CCTTAAACAGSA | 292 | CTTTTCAAATSC |
| | | | | | ATTTGAAAAG | | TGTTTAAGG | | TTTGAAAAG | | TGTTTAAGG |
| MRP1 | C > A | 1625 | U07050 | 293 | GGGAATCACTA | 294 | CAGAGAGGTTT | 295 | GGGAATCACTM | 296 | CAGAGAGGTTK |
| | | | | | AACCTCTCTG | | AGTGATTCCC | | AACCTCTCTG | | AGTGATTCCC |
| MRP1 | C > T | 1163 | U07050 | 297 | TGTGATCGGCT | 298 | AGCCGAGGCGA | 299 | TGTGATCGGCY | 300 | AGCCGAGGCGR |
| | | | | | CGCCTCGGCT | | GCCGATCACA | | CGCCTCGGCT | | GCCGATCACA |
| MRP1 | A > G | 381 | U07050 | 301 | TGGGGGACCCG | 302 | TTTATTGGCCC | 303 | TGGGGGACCCR | 304 | TTTATTGGCCYG |
| | | | | | GGCCAATAAA | | GGGTCCCCCA | | GGCCAATAAA | | GGTCCCCCA |
| MRP1 | G > A | 233 | U07050 | 305 | AAGAGTAGCAA | 306 | CAAGATAAAATT | 307 | AAGAGTAGCAR | 308 | CAAGATAAAAYT |
| | | | | | TTTTATCTTG | | GCTACTCTT | | TTTTATCTTG | | GCTACTCTT |
| MRP1 | C > A | 189 | U07050 | 309 | AAAAAAATCCAA | 310 | TTTTTGGATTTG | 311 | AAAAAAATCCM | 312 | TTTTTGGATTKG |
| | | | | | ATCCAAAAA | | GATTTTTTT | | AATCCAAAAA | | GATTTTTTT |
| MRP1 | C > T | 440 | U07050 | 313 | CTCCTTCCCTTG | 314 | AGGACCTAGCA | 315 | CTCCTTCCCTY | 316 | AGGACCTAGCR |
| | | | | | CTAGGTCCT | | AGGGAAGGAG | | GCTAGGTCCT | | AGGGAAGGAG |
| MRP1 | de/AT | 34206 | AC003026 | 317 | AGTCTCACACG | 318 | GTGAGTGCACG | 319 | AGTCTCACACn | 320 | GTGAGTGCACn |
| | | to | | | TGCACTCAC | | TGTGAGACT | | GTGCACTCAC | | GTGTGAGACT |
| | | 34207 | | | | | | | | | |
| MRP1 | de/GG | 1720 to | U07050 | 321 | ACTCCAGGCAG | 322 | GAACGGAGCCT | 323 | ACTCCAGGCAn | 324 | GAACGGAGCCn |
| | TA | 1723 | | | GCTCCGTTC | | GCCTGGAGT | | GGCTCCGTTC | | TGCCTGGAGT |
| MRP1 | InsT | 926/ | U07050 | 325 | TTAATTTTTTTTT | 326 | AAATAATAATAA | 327 | TTAATTTTTTTTn | 328 | AAATAATAATnA |
| | | 927 | | | ATTATTATTT | | AAAAAAATTAA | | ATTATTATTT | | AAAAAAATTAA |
| MRP1 | InsTC | 437/ | U07050 | 329 | TTCCTCCTTCCT | 330 | ACCTAGCGAGG | 331 | TTCCTCCTTCCn | 332 | ACCTAGCGAGA |
| | CTTC | 438 | | | CCTTCCCTCGC | | GAAGGAGGAAG | | CTCGCTAGGT | | GGAAGGAGGAA |
| | C | | | | TAGGT | | GAGGAA | | | | |
| MRP1 | insTG | 55156/ | AC003026 | 333 | GGGGCTGGGG | 334 | CACGCACCCGA | 335 | GGGGCTGGGG | 336 | CACGCACCCGn |
| | GGG | 55157 | | | CTGGGGCTGGG | | CCCCGACCCAG | | CnTGGGTGCGT | | ACCCAGCCCC |
| | C | | | | TGCGTG | | CCCC | | G | | |
| MDR1 | T > C | 140837 | AC002457 | 337 | GCTCATTCGAG | 338 | AGAGCCGCTGC | 339 | CTCATTCGAGY | 340 | AGAGCCGCTRC |
| | | | | | CAGCGGCTCT | | TCGAATGAG | | AGCGGCTCTT | | TCGAATGAG |
| MDR1 | G > A | 84701 | AC005068 | 341 | AAAATTGCTATC | 342 | AGATAGTGATA | 343 | AAAATTGCTRTC | 344 | AGATAGTGAYA |
| | | | | | ACTATCT | | GCAATTTT | | ACTATCT | | GCAATTTT |
| MDR1 | G > A | 101 | M29432 | 345 | TTCACTTCAATT | 346 | ATGGGTAATTG | 347 | TCACTTCARTTA | 348 | GATGGGTAAYT |
| | | | | | ACCCATC | | AAGTGAA | | CCCATC | | GAAGTGAA |
| MDR1 | C > T | 308 | M29432 | 349 | CTTGAAGGGTC | 350 | TCAGGTTCAGA | 351 | TCTTGAAGGGY | 352 | TCAGGTTCAGR |
| | | | | | TGAACCTGA | | CCCTTCAAGA | | CTGAACCTG | | CCCTTCAAGA |
| MDR1 | C > T | 83946 | AC005068 | 353 | TCAGCAGTTAC | 354 | TGCAATGTAACT | 355 | CAGCAGTYACA | 356 | TGCAATGTRACT |
| | | | | | ATTGCA | | GCTGA | | TTGCAC | | GCTGA |
| MDR1 | G > A | 83973 | AC005068 | 357 | GACCCATGCAA | 358 | GGTCTAGCTTG | 359 | GACCCATGCRA | 360 | GGTCTAGCTYG |
| | | | | | GCTAGACC | | CATGGGTC | | GCTAGACC | | CATGGGTC |
| MDR1 | A > G | 84032 | AC005068 | 361 | GAGCACAACGG | 362 | CAGCTGGACCG | 363 | GAGCACAACRG | 364 | CAGCTGGACYG |
| | | | | | TCCAGCTG | | TTGTGCTC | | TCCAGCTG | | TTGTGCTC |
| MDR1 | G > A | 84074 | AC005068 | 365 | TGGGCAGACAG | 366 | CAGGGCCACTG | 367 | TGGGCAGACRG | 368 | CAGGGCCACYG |
| | | | | | TGGCCCTG | | TCTGCCCA | | TGGCCCTG | | TCTGCCCA |
| MDR1 | G > A | 84119 | AC005068 | 369 | CTCGTCCTGAT | 370 | CAAGATCTATCA | 371 | CTCGTCCTGRT | 372 | CAAGATCTAYCA |
| | | | | | AGATCTTG | | GGACGAG | | AGATCTTG | | GGACGAG |
| MDR1 | A > G | 77811 | AC005068 | 373 | GGCTTGAAGGT | 374 | ATTCTTACACCT | 375 | GGCTTGAAGRT | 376 | ATTCTTACAYCT |
| | | | | | GTAAGAAT | | TCAAGCC | | GTAAGAAT | | TCAAGCC |
| MDR1 | T > A | 78170 | AC005068 | 377 | TATTCCTTTACA | 378 | CAAAAATTTGTA | 379 | TATTCCTTTACW | 380 | ACAAAAATTWG |
| | | | | | AATTTTTG | | AAGGAATA | | AATTTTTG | | TAAAGGAAT |
| MDR1 | A > G | 73252 | AC005068 | 381 | ACTTTGTCTGAT | 382 | GCAGGAGATCA | 383 | ACTTTGTCTRAT | 384 | GCAGGAGATYA |
| | | | | | CTCCTGC | | GACAAAGT | | CTCCTGC | | GACAAAGT |
| MDR1 | G > A | 141529 | A0002457 | 385 | CTTCAGGTCGG | 386 | CAAGATCCATTC | 387 | CTTCAGGTCGG | 388 | CAAGATCCATY |
| | | | | | AATGGATCTTG | | CGACCTGA | | RATGGATCTTG | | CCGACCTGAAG |
| MDR1 | A > G | 141590 | AC002457 | 389 | AAACTGAACGA | 390 | TACCTTTTATCG | 391 | AAACTGAACRAT | 392 | TACCTTTTATYG |
| | | | | | TAAAAGGTA | | TTCAGTTTAA | | AAAAGGTA | | TTCAGTTTAA |
| MDR1 | C > T | 70200 | AC005068 | 393 | TTCTCCTTATGG | 394 | CTAACACCCAT | 395 | TTCTCCTTAYGG | 396 | CTAACACCCRT |
| | | | | | GTGTTAG | | AAGGAGAA | | GTGTTAG | | AAGGAGAA |
| MDR1 | C > A | 70204 | AC005068 | 397 | AATTTCTCATT | 398 | CACCCGTAATG | 399 | AATTTTCTCMTT | 400 | CACCCGTAAKG |
| | | | | | ACGGGTG | | AGAAAATT | | ACGGGTG | | AGAAAATT |
| MDR1 | C > T | 70237 | AC005068 | 401 | TTAATTGGCTAT | 402 | GTCCAAAATAG | 403 | TTAATTGGCYAT | 404 | GTCCAAAATRG |
| | | | | | TTTGGAC | | CCAATTAA | | TTTGGAC | | CCAATTAA |
| MDR1 | G > A | 70253 | AC005068 | 405 | TCTACTGGTATT | 406 | TAAGACAAATAC | 407 | TCTACTGGTRTT | 408 | TAAGACAAAYAC |
| | | | | | TGTCTTA | | CAGTAGA | | TGTCTTA | | CAGTAGA |
| MDR1 | C > A | 70371 | AC005068 | 409 | AATCATTTTATG | 410 | TGTGGCACATA | 411 | AATCATTTTMTG | 412 | TGTGGCACAKA |
| | | | | | TGCCACA | | AAATGATT | | TGCCACA | | AAATGATT |
| MDR1 | C > T | 137 | M29445 | 413 | GAACATTGCTTA | 414 | GTCTCCATAAG | 415 | GAACATTGCYTA | 416 | GTCTCCATARG |
| | | | | | TGGAGAC | | CAATGTTC | | TGGAGAC | | CAATGTTC |
| MDR1 | C > T | 176 | M29445 | 417 | GAAGAGATTGT | 418 | CCCTCACAATC | 419 | GAAGAGATYGT | 420 | CCCTCACRATC |
| | | | | | GAGGG | | TCTTC | | GAGGGC | | TCTTC |
| MDR1 | A > C | 43263 | AC005068 | 421 | TGAATGTTCCGT | 422 | CGGAGCCACGG | 423 | TGAATGTTCMG | 424 | CGGAGCCACKG |
| | | | | | GGCTCCG | | AACATTCA | | TGGCTCCG | | AACATTCA |
| MDR1 | T > A | 43162 | AC005068 | 425 | CGGGTGGTGAC | 426 | CTTCCTGTGTCA | 427 | CGGGTGGTGW | 428 | CTTCCTGTGWC |
| | | | | | ACAGGAAG | | CCACCCG | | CACAGGAAG | | ACCACCCG |
| MDR1 | C > T | 145984 | AC002457 | 429 | AAAATACTTTGG | 430 | CAAATTTCCAAA | 431 | AAAATACTTYGG | 432 | CAAATTTCCRAA |
| | | | | | AAATTTG | | GTATTTT | | AAATTTG | | GTATTTT |
| MDR1 | T > C | 171404 | AC002457 | 433 | ATCATTAAACGA | 434 | ACTCATTTCGTT | 435 | ATCATTAAAYGA | 436 | ACTCATTTCRTT |
| | | | | | AATGAGT | | TAATGAT | | AATGAGT | | TAATGAT |
| MDR1 | G > C | 171456 | AC002457 | 437 | GACTAAAGACA | 438 | CATTTTATGTGTC | 439 | GACTAAAGASA | 440 | CATTTATGTSTC |
| | | | | | CATAAATG | | TTTAGTC | | CATAAATG | | TTTAGTC |
| MDR1 | G > T | 171466 | AC002457 | 441 | GACATAAATGTT | 442 | AAACAAACATAA | 443 | AGACATAAATG | 444 | AAACAAACATA |
| | | | | | ATGTTTGTTT | | CATTTATGTCT | | KTATGTTTGT | | MCATTTATGTC |
| MDR1 | T > C | 171511 | AC002457 | 445 | GATACAGGGCT | 446 | TCATGAAGAGC | 447 | GATACAGGGYT | 448 | TCATGAAGARC |
| | | | | | CTTCATGA | | CCTGTATC | | CTTCATGA | | CCTGTATC |
| MDR1 | T > C | 171512 | AC002457 | 449 | GATACAGGGTC | 450 | ATTCATGAAGG | 451 | GATACAGGGTY | 452 | ATTCATGAAGRA |
| | | | | | CTTCATGAAT | | ACCCTGTATC | | CTTCATGAAT | | CCCTGTATC |
| MDR1 | G > A | 174901 | AC002457 | 453 | GTGCACGATAT | 454 | GCTCCCCAATA | 455 | GTGCACGATRT | 456 | GCTCCCCAAYA |
| | | | | | TGGGGAGC | | TCGTGCAC | | TGGGGAGC | | TCGTGCAC |
| MDR1 | C > T | 175068 | AC002457 | 457 | TAAGCAGCAAT | 458 | ACAGGACATTAT | 459 | TAAGCAGCAAY | 460 | ACACGACATTRT |
| | | | | | AATGTCGTGT | | TGCTGCTTA | | AATGTCGTGT | | TGCTGCTTA |
| MDR1 | C > T | 175074 | AC002457 | 461 | CAACAATGTTGT | 462 | GATGCACACAA | 463 | CAACAATGTYGT | 464 | GATGCACACRA |
| | | | | | GTGCATC | | CATTGTTG | | GTGCATC | | CATTGTTG |
| MDR1 | A > G | 175142 | AC002457 | 465 | CATTAAATGGA | 466 | CCCAGTCCTCC | 467 | CATTAAATGRAG | 468 | CCCAGTCCTYC |
| | | | | | GGACTGGG | | ATTTAATG | | GACTGGG | | ATTTAATG |
| MDR1 | A > G | 175180 | AC002457 | 469 | TCCTCTGAGGA | 470 | ACTGCACATCC | 471 | TCCTCTGAGRA | 472 | ACTGCACATYCT |
| | | | | | TGTGCAGT | | TCAGAGGA | | TGTGCAGT | | CAGAGGA |
| MDR1 | A > G | 139015 | AC002457 | 473 | AACTTACTTGTA | 474 | TCAAAGATACAA | 475 | AACTTACTTRTA | 476 | TCAAAGATAYAA |
| | | | | | TCTTTGA | | GTAAGTT | | TCTTTGA | | GTAAGTT |
| MDR1 | A > T | 139064 | AC002457 | 477 | AGAAATAGTTTA | 478 | TGTTGATTAAAC | 479 | AGAAATAGTWT | 480 | TGTTGATTAWA |
| | | | | | ATCAACA | | TATTTCT | | AATCAACA | | CTATTTCT |
| MDR1 | T > C | 139119 | AC002457 | 481 | TAGGGAGGGCT | 482 | TGGCCTTAAGC | 483 | TAGGGAGGGYT | 484 | TGGCCTTAARC |
| | | | | | TAAGGCCA | | CCTCCCTA | | TAAGGCCA | | CCTCCCTA |
| MDR1 | G > A | 139177 | AC002457 | 485 | GAAAGGTGAAA | 486 | TTGCTTTATTTC | 487 | GAAAGGTGARA | 488 | TTGCTTTATYTC |
| | | | | | TAAAGCAA | | ACCTTTC | | TAAAGCAA | | ACCTTTC |
| MDR1 | C > T | 139276 | AC002457 | 489 | CATTTACCCTAG | 490 | GGTCCATCTAG | 491 | CATTTACCCYAG | 492 | GGTCCATCTRG |
| | | | | | ATGGACC | | GGTAAATG | | ATGGACC | | GGTAAATG |
| MDR1 | G > A | 140118 | AC002457 | 493 | ATATGGAAGAA | 494 | TTGTAATTTTCT | 495 | ATATGGAAGRA | 496 | TTGTAATTTYCT |
| | | | | | AATTACAA | | TCCATAT | | AATTACAA | | TCCATAT |
| MDR1 | A > G | 140216 | AC002457 | 497 | AACACGGGCGT | 498 | TCAGATCAACG | 499 | AACACGGGCRT | 500 | TCAGATCAAYG |
| | | | | | TGATCTGA | | CCCGTGTT | | TGATCTGA | | CCCGTGTT |
| MDR1 | T > C | 140490 | A0002457 | 501 | TGTATTAAACGC | 502 | GGGATTCGCGT | 503 | TGTATTAAAYGC | 504 | GGGATTCGCRT |
| | | | | | GAATCCC | | TTAATACA | | GAATCCC | | TTAATACA |
| MDR1 | G > A | 140568 | AC002457 | 505 | TTGAAAGACAT | 506 | ATGTAGACATGT | 507 | TTGAAAGACRT | 508 | ATGTAGACAYG |
| | | | | | GTCTACAT | | CTTTCAA | | GTCTACAT | | TCTTTCAA |
| MDR1 | A > T | 140576 | AC002457 | 509 | CGTGTCTACTTA | 510 | TTCAACTTAAGT | 511 | CGTGTCTACWT | 512 | TTCAACTTAWG |
| | | | | | AGTTGAA | | AGACACG | | AAGTTGAA | | TAGACACG |
| MDR1 | A > G | 140595 | AC002457 | 513 | ATGTCCCCAGT | 514 | GCTGAATCACT | 515 | ATGTCCCCART | 516 | GCTGAATCAYT |
| | | | | | GATTCAGC | | GGGGACAT | | GATTCAGC | | GGGGACAT |
| MDR1 | G > A | 140727 | AC002457 | 517 | CCGGGCCGGAA | 518 | ATGACTGCTTC | 519 | CCGGGCCGGRA | 520 | ATGACTGCTYC |
| | | | | | GCAGTCAT | | CGGCCCGG | | GCAGTCAT | | CGGCCCGG |
| MDR1 | G > A | 139479 | AC002457 | 521 | GAGGCGGGCA | 522 | CTCGTGATCTG | 523 | GAGGCGGGCR | 524 | CTCGTGATCYG |
| | | | | | GATCACGAG | | CCCGCCTC | | GATCACGAG | | CCCGCCTC |
| MDR1 | T > C | 139619 | AC002457 | 525 | GGAGAATGGCG | 526 | CGGGTTCACGC | 527 | GGAGAATGGYG | 528 | CGGGTTCACRC |
| | | | | | TGAACCCG | | CATTCTCC | | TGAACCCG | | CATTCTCC |
| MDR1 | G > T | 65241 | AC005068 | 636 | ACTAGAAGGTT | 637 | ACCTTCCCAGA | 638 | ACTAGAAGGTK | 639 | ACCTTCCCAGM |
| | | | | | CTGGGAAGGT | | ACCTTCTAGT | | CTGGGAAGGT | | ACCTTCTAGT |
| MDR1 | G > A | 50537 | AC005068 | 640 | TCCTGACTATAC | 641 | TTGGCTTTGGTA | 642 | TCCTGACTATRC | 643 | TTGGCTTTGGY |
| | | | | | CAAAGCCAA | | TAGTCAGGA | | CAAAGCCAA | | ATAGTCAGGA |
| TOP1 | 1334 | 133418 | GI:11225259 | 529 | ACTTTTCCGTTG | 530 | TTGCCGCGGCA | 531 | ACTTTTCCGTKG | 532 | TTGCCGCGGCM |
| | G > T | 45 | | | CCGCGGCAACT | | ACGGAAAAGTTC | | CCGCGGCAACT | | ACGGAAAAGTTC |
| TOP1 | 1845 | 1845 | GI:11225259 | 533 | CTCGGGAAGGG | 534 | TCTGATGGAGC | 535 | CTCGGGAAGGR | 536 | TCTGATGGAGY |
| | A > G | | | | CTCCATCAGA | | CCTTCCCGAG | | CTCCATCAGA | | CCTTCCCGAG |

TABLE 2
The nucleic acid and amino acid sequences referred to in this application

| | | Protein Acc | SEQ | | SEQ | |
|---|---|---|---|---|---|---|
| Gene | AS change | No | ID NO | Protein | ID N= | Protein wt > mut |
| UGT1A1 | L15R | G8850236 | 538 | PLVLGRLLCVL | 539 | PLVLGXLLCVL |
| UGT1A1 | G71R | G8850236 | 540 | LYIRDRAFYTL | 541 | LYIRDXAFYTL |
| UGT1A1 | D119Dframeshift | G8850236 | 542 | KKIKKDCYAFC | 543 | KKIKKDX |
| UGT1A1 | P152Pframeshift | G8850236 | 544 | VMLTDPFPSLQ | 545 | VMLTDPX |
| UGT1A1 | F170del | G8850236 | 546 | LSLPTVFLHAL | 547 | LSLPTVFX |
| UGT1A1 | L175Q | G8850236 | 548 | FFLHAQPCSLE | 549 | FFLHAXPCSLE |
| UGT1A1 | C177R | G8850236 | 550 | LHALPRSLEFE | 551 | LHALPXSLEFE |
| UGT1A1 | R209W | G8850236 | 552 | MTFLQWVKNML | 553 | MTFLQXVKNML |
| UGT1A1 | P229Q | G8850236 | 554 | DVVYSQYATLA | 555 | DVVYSXYATLA |
| UGT1A1 | G276R | G8850236 | 556 | NMVFVRGINCL | 557 | NMVFVXGINCL |
| UGT1A1 | A292V | G8850236 | 558 | SQEFEVYINAS | 559 | SQEFEXYINAS |
| UGT1A1 | Y293Wframeshift | G8850236 | 560 | QEFEAWRTWN | 561 | QEFEAXINASG |
| UGT1A1 | G308E | G8850236 | 562 | VVFSLESMVSE | 563 | VVFSLXSMVSE |
| UGT1A1 | Q331R | G8850236 | 564 | LGKIPRTVLWR | 565 | LGKIPXTVLWR |
| UGT1A1 | Q357R | G8850236 | 566 | VKWLPRNDLLG | 567 | VKWLPXNDLLG |
| UGT1A1 | R367G | G8850236 | 568 | GHPMTGAFITH | 569 | GHPMTXAFITH |
| UGT1A1 | A368T | G8850236 | 570 | HPMTRTFITHA | 571 | HPMTRXFITHA |
| UGT1A1 | P387R | G8850236 | 572 | ICNGVRMVMMP | 573 | ICNGVXMVMMP |
| UGT1A1 | S375F | G8850236 | 574 | ITHAGFHGVYE | 575 | ITHAGXHGVYE |
| UGT1A1 | S381R | G8850236 | 576 | HGVYERICNGV | 577 | HGVYEXICNGV |
| UGT1A1 | A401P | G8850236 | 578 | DQMDNPKRMET | 579 | DQMDNXKRMET |
| UGT1A1 | R403Rframeshift | G8850236 | 580 | MDNAKRHGD. | 581 | MDNAKX |
| UGT1A1 | K428E | G8850236 | 582 | LENALEAVIND | 583 | LENALXAVIND |
| UGT1A1 | Y486D | G8850236 | 584 | LTWYQDHSLDV | 585 | LTWYQXHSLDV |
| UGT1A1 | S488F | G8850236 | 586 | WYQYHFLDVIG | 587 | WYQYHXLDVIG |
| UGT1A1 | Q49stop | G8850236 | 588 | LGAIQ. | 589 | LGAIQ. |
| UGT1A1 | C280stop | G8850236 | 590 | VGGIN. | 591 | VGGIN. |
| UGT1A1 | Q331stop | G8850236 | 592 | LGKIP. | 593 | LGKIP. |
| UGT1A1 | W335stop | G8850236 | 594 | PQTVL. | 595 | PQTVL. |
| UGT1A1 | Q357stop | G8850236 | 596 | VKWLP. | 597 | VKWLP. |
| UGT1A1 | K437stop | G8850236 | 598 | NDKSY. | 599 | NDKSY. |
| MRP1 | F329C | G2828206 | 600 | YFLMSCFFKAI | 601 | YFLMSXFFKAI |
| MRP1 | R433S | G2828206 | 602 | SVDAQSFMDLA | 603 | SVDAQXFMDLA |
| MRP1 | R723Q | G2828206 | 604 | QNDSLQENILF | 605 | QNDSLXENILF |
| MDR1 | N21D | G2506118 | 606 | FFKLNDKSEKD | 607 | FFKLNXKSEKD |
| MDR1 | F103L | G2506118 | 608 | INDTGLFMNLE | 609 | INDTGXFMNLE |
| MDR1 | V168I | G2506118 | 610 | FDVHDIGELNT | 611 | FDVHDXGELNT |
| MDR1 | S400N | G2506118 | 612 | RNVHFNYPSRK | 613 | RNVHFXYPSRK |
| MDR1 | G412G | G2506118 | 614 | VKILKGLNLKV | 615 | VKILKXLNLKV |
| MDR1 | T436T | G2506118 | 616 | CGKSTTVQLMQ | 617 | CGKSTXVQLMQ |
| MDR1 | A893S | G2506118 | 618 | KELEGSGKIAT | 619 | KELEGXGKIAT |
| MDR1 | A999T | G2506118 | 620 | FAPDYTKAKIS | 621 | FAPDYXKAKIS |
| MDR1 | A1001T | G2506118 | 622 | PDYAKTKISAA | 623 | PDYAKXKISAA |
| MDR1 | Q1107P | G2506118 | 624 | KRLNVPWLRAH | 625 | KRLNVXWLRAH |
| MDR1 | A1132A | G2506118 | 626 | IAENIAYGDNS | 627 | IAENIXYGDNS |
| MDR1 | S1141T | G2506118 | 628 | NSRVVTQEEIV | 629 | NSRVVXQEEIV |
| MDR1 | I1145I | G2506118 | 630 | VSQEEIVRAAK | 631 | VSQEEXVRAAK |
| TOP1 | G363C | G12644118 | 632 | PGLFRCRGNHP | 633 | PGLFRXRGNHP |
| TOP1 | D533G | G12644118 | 634 | DFLGKGSIRYY | 635 | DFLGKXSIRYY |

### THE FIGURES SHOW

FIG. 1 shows the correlation of the exon 26 SNP with intestinal MDR1 expression in 21 volunteers determined by Western Blot analyses. The box plot shows the distribution of MDR1 expression clustered according to the MDR1 3435>T genotype at position corresponding to position 176 of the MDR1 gene (GenBank Acc. No. M29445). The T allele was associated with a lower expression of p-glycoprotein.

FIG. 2 shows the correlation of MDR1 3435C>T genotype and digoxin uptake in 14 healthy volunteers who participated in a clinical study that addresses peak plasma levels of digoxin at steady state [Johne et al., 1999, Clin. Pharmacol. Ther. 66:338-345]. Maximum digoxin levels were statistically significantly different (p=0.006, Mann Whitney U test) between the two groups which were homozygous for the T and C allele, respectively.

FIG. 3 represents the correlation of the genotype (wt/wt: 1; wt/mut and mut/mut:2) with MRP1 mRNA content in duodenal biopsies from healthy volunteers derived from two independent experiments, before and after application of rifampicin. Treatment with rifampicin had no effect on MRP1 mRNA expression (p<0.001, paired t-test). A strong trend of an association of MRP1 genotype with MRP1 mRNA levels was detected (p=0.086, Kruskal-Wallis test).

FIGS. 4 to 28 show the nucleic acid and amino acid sequences referred to herein.

FIG. 29 shows the expression profile of genes relevant to Irinotecan metabolism in carcinoma cell lines. This semiquantitativ RT-PCR shows amounts of transcripts for the genes indicated right to the amplicons. PCR products were analyzed by agarose electrophoresis, stained with ethidium bromid. The respective fragment sizes are indicated on the left in basepaires (bp).

FIG. 30 shows growth inhibition curves for CPT-11 (A) and SN-38 (B) with epithelial carcinoma cell lines LS174T (colon), KB 3-1 (cervix) and RT112 (bladder). Concentrations of CPT-11 ranged from 0 to 200 μg/ml and of SN-38 from 0 to 200 ng/ml. Cells were treated for three days. The data for each concentration are mean values of at least three wells.

FIG. 31 growth inhibition curves for CPT-11 (A) and SN-38 (B) with a epithelial cervix carcinoma cell line KB 3-1 and two subclones expressing high amounts of MDR1, KB 3-1 (MDR1) and KB 3-1 (MDR1, CYP3A5). Concentrations of CPT-11 ranged from 0 to 200 μg/ml and of SN-38 from 0 to 200 ng/ml. Cells were treated for three days. The data for each concentration are mean values and standard deviation of at least three wells.

FIG. 32 shows growth inhibition curves for CPT-11 (A) and SN-38 (B) with the bladdercancer cell line RT112 and and its subclones RT112 (MDR1, UGT1A1) expressing MDR1 and higher amounts of UGT1A1. Concentrations of CPT-11 ranged from 0 to 200 μg/ml and of SN-38 from 0 to 200 ng/ml. Cells were treated for three days. The data for each concentration are mean values and standard deviation of at least three wells.

FIG. 33 shows growth inhibition curves for CPT-11 (A) and SN-38 (B) with inhibition of MDR1 by R-Verapamil. The epithelial cervix carcinoma cell line KB 3-1 and the two subclones KB 3-1 (MDR1) and KB 3-1 (MDR1, CYP3A5), with high MDR1 expression, were tested for the influence of MDR1 inhibition by R-Verapamil on drug sensitivity. Concentrations of CPT-11 ranged from 0 to 200 μg/ml and of SN-38 from 0 to 200 ng/ml and R-Verapamil was added to 10 μg/ml final concentration(+V). Cells were treated for three days. The data for each concentration are mean values of two wells.

FIG. 34 shows growth inhibition curves for CPT-11 (A) and SN-38 (B) with inhibition of MDR1 by R-Verapamil. To circumvent the MDR1 effect on drug resistance cells were treated in parallel with R-Verapamil. The KB 3-1 (MDR1) and KB 3-1 (MDR1, CYP3A5), which differ in their CYP3A5 expression, were tested for remaining resistance after inhibition of MDR1. Concentrations of CPT-11 ranged from 0 to 200 μg/ml and of SN-38 from 0 to 200 ng/ml and R-Verapamil was added to 10 μg/ml final concentration(+V). Cells were treated for three days. The data for each concentration are mean values of two wells.

The present invention is illustrated by reference to the following biological Examples which are merely illustrative and are not to be constructed as a limitation of the scope of the present invention.

### EXAMPLE 1

### Phenotypically Impact of the C to T Substitution at Position Corresponding to Position 176 of the MDR1 Gene (Acc. No. M29445)

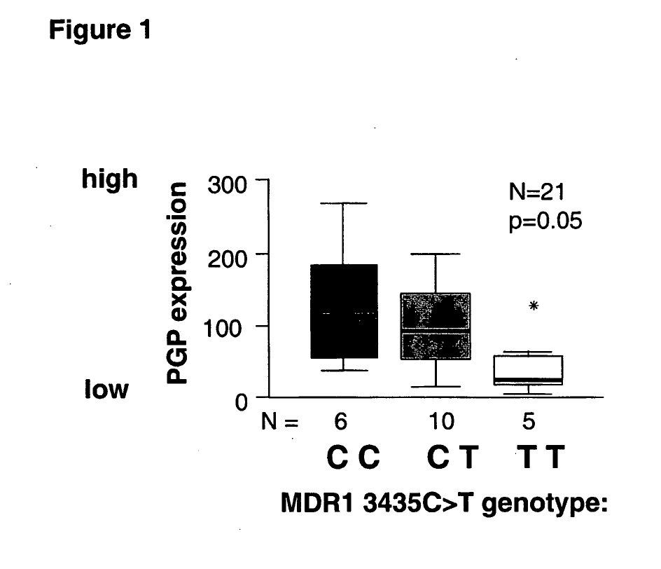

To investigate the influence of the single nucleotide C to T substitution at position corresponding to position 176 of the MDR1 gene (Acc. No. M29445) also referred to as MDR1 exon 26 SNP C3435T on intestinal P-glycoprotein (PGP) expression, samples from biopsies and duodenal enterocyte preparations from 21 were investigated at the Dr. Margarete Fischer-Bosch-Institute for Clinical Pharmacology in Stuttgart by quantitative immunohistochemistry and Western blots. The results are shown in FIG. 1. Homozygous carriers of the T allele (having at a position corresponding to position 176 of the MDR1 gene (Accession No: M29445) a T) demonstrated significantly higher PGP levels compared to homozygous carriers of the C allele (having at a position corresponding to position 176 of the MDR1 gene (Accession No: M29445) a C). Individuals with heterozygous genotype showed an intermediate level of PGP expression.

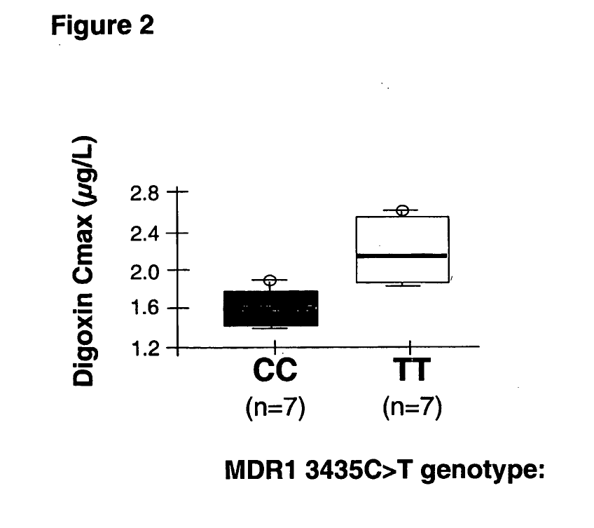

Furthermore, the influence of the MDR1 genotype on intestinal uptake-related pharmacokinetics of digoxin was investigated in a clinical study at the University Medical Center, Charite in Berlin. Maximal digoxin blood levels (Cmax) at steady state were correlated with the MDR1 3435C>T genotype 14 healthy volunteers after oral application of digoxin. FIG. 2 shows, volunteers homozygous for the T allele show statistically significantly lower digoxin levels than volunteers with a C/C genotype. (p=0.006, Mann Whitney U test) and reflects the impact of this polymorphism on digoxin pharmacokinetics.

### EXAMPLE 2

### Correlation of MRP1 Polymorphisms with MRP1 Expression and Side Effects During Therapy with MRP1 Substrates

Functional polymorphisms in the MRP1 gene affect the transport activity which in consequence modulates plasma levels and/or intracellular concentrations of MRP1 substrate drugs. Increased levels of such drugs can lead to side effects whereas decreased levels may result in subtherapeutical drug levels and therapy failure. MRP1 polymorphisms were correlated with the occurence of drug-related adverse effects and therapeutic efficacy in patients treated with MRP1 substrate drugs. In a case-control study, the frequency distribution of MRP1 SNPs was compared between a group of patients who suffered from cisplatin-related nephrotoxicity and a group of patients with nephro- and hepatotoxicities caused from anti-cancer drugs with a group of healthy controls. Furthermore, samples of known MRP1 mRNA levels were screened for MRP1 genotype. The results in the group of patients demonstrating nephro- and hepatotoxicity during anti-cancer treatment, are listed in the following table for one MRP1 SNP:

| | | Allele | | Genotype frequency [%] | | |
|---|---|---|---|---|---|---|
| | | frequency [%] | | | | *A/A |
| SNP | group | G allele | A allele | *G/A | *A/A | expected² |
| 150727G > A¹ | Controls | 66.7 | 33.3 | 50 | 8.3 | 10.9 |
| | Cases | 50.0 | 50.0 | 14.3 | 42.9 | 25.0 |

¹according to Acc. No. AC025277
²calculated according to Hardy-Weinberg

In contrast to control samples, the A allele (substitution of G to A at position according to position 150727 of the MRP1 gene, Acc. No. AC025277) was statistically significantly overrepresented in patients suffering from drug-related kidney- and liver side effects compared to healthy controls (p=0.044, Chi² test) and was thus predictive for these side effects.

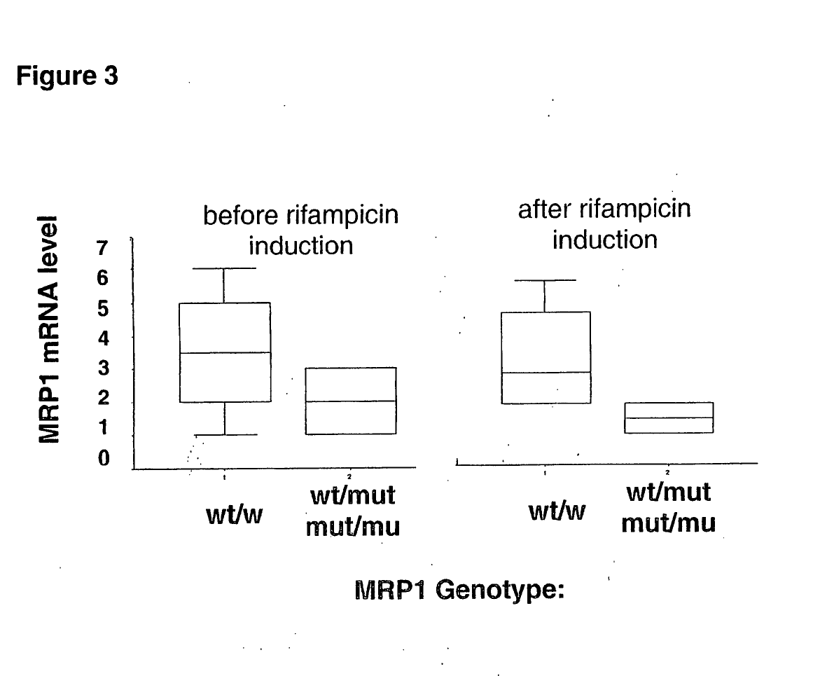

Furthermore, an association of MRP1 genotype with mRNA expression before and after rifampicin application was detected for two MRP1 SNP's, 95T>C (SEQ ID NOs. 209, 210, 211, and 212, nucleotide substitution of T to C at a position corresponding to position 95 of the MRP1 gene, Acc. No. AF022831) and 259A>G (SEQ ID NOs. 277, 278, 279, and 280, nucleotide substitution of A to G at a position corresponding to position 259 of the MRP1 gene, Acc. No. AF022831). These SNPs are linked and form one allele. The mutant allele (MRP1mut, C at position 95 and G at position 259 of the MRP1 gene, Acc. No. AF022831) is statistically significantly correlated with decreased MRP1 mRNA expression and the wildtype allele (MRP1wt, T at position 95 and A at position 259 of the MRP1 gene, Acc. No. AF022831) with increased MRP1 expression in two independent experiments (with and without rifampicin induction), as illustrated in FIG. 3.

The differences in the MRP1 mRNA content are based on MRP1 genotype-related interindividual differences and the analysis of these SNP's is of high diagnostic and prognostic value for MRP1 expression levels and to predict the therapeutic outcome and adverse effects of MRP1 substrate drugs.

### EXAMPLE 3

### Dosage Calculation

Therapeutic efficacy ans adverse effects of irinotecan depend on plasma levels and intracellular concentrations of the parent compound and the active metabolites (e.g. SN-38), processes which are controlled by CYP3A5- and UGT1A1-related metabolism and MRP1- and MDR1-related transport processes [Atsumi, et al., 1991, Xenobiotica 21:1159-69, lyer, et al., 1998, J Clin Invest 101:847-54, Ciotti, et al., 1999, Biochem Biophys Res Commun 260:199-202, Santos, et al., 2000, Clin Cancer Res 6:2012-20, Kuhn, 1998, Oncology (Huntingt) 12:3942, Chen, et al., 1999, Mol Pharmacol 55:921-8, Chu, et al., 1997, Cancer Res 57:1934-8, Chu, et al., 1997, J Pharmacol Exp Ther 281:304-14; Chu, et al., 1998, Cancer Res 58:5137-43, Chu, et al., 1999, Drug Metab Dispos 27:440-1, Chu, et al., 1999, J Pharmacol Exp Ther 288:735-41, Mattern, et al., 1993, Oncol Res 5:467-74, Hoki, et al., 1997, Cancer Chemother Pharmacol 40:433-8, Sugiyama, et al., 1998, Cancer Chemother Pharmacol 42:S44-9]. For example, MRP1 works in close connection with glucuronosyltransferases as part of the cellular detoxification system and is known to transport glucuronosyl conjugates such as SN-38G [König et al., 1999, Biochim Biophys Acta 1461:377-394, Kerb et al., 2001, Pharmacogenomics 2:51-64]. For example, the extend to which SN-38G is exported from the cell into bile greatly influences the rate of its formation. For an efficient detoxification of SN-38 both processes are necessary, conjugation by UGT1A1 and export of the glucuronide.

The 47518T>C (SEQ ID NOs.137, 138, 139, and 140) and 9736A>G (SEQ ID NOs. 149, 150, 151, 152) nucleotide substitutions of the CYP3A5 gene (Acc. No. GI:10281451), and the 145601T>G (SEQ ID NOs. 141, 142, 143, 144) and 145929A>G (SEQ ID NOs. 145, 146, 147, and 148) nucleotide substitutions of the CYP3A5 gene (Acc. No. GI:11177452) form an high CYP3A5 expression-related allele and are therefore associated with a higher metabolic inactivation of irinotecan. Individuals with this allele are extensive metabolizers (EMs) and are therefore in contrast the reminder poor metabolizers (PMs) less likely to suffer from irinotecan toxicity. Those with one high expressor and one low expressor-related allele are regarded as intermediate metabolizers (IMs).

The 176C>T nucleotide substitution (SEQ ID NOs. 217, 218, 219, and 220) of the MDR1 gene (Accession No: M29445) is associated with low PGP expression-related low drug efflux, and the 95T>C (SEQ ID NOs. 209, 210, 211, and 212) and the 259A>G (SEQ ID NOs. 277, 278, 279, and 280) nucleotide substitutions of the MRP1 gene (Acc. No. AF022831) are associated with low mRNA expression and the 150727G>A nucleotide substitution (SEQ ID NOs. 217, 218, 219, and 220) of the MRP1 gene (Accession No: M29445) is associated with low PGP expression-related low drug efflux and the 150727G>A nucleotide substitution (SEQ ID NOs. 217, 218, 219, and 220) of the MRP1 gene (Accession No: AC025277) is associated with adverse effects. Individuals carrying low transporter expression-related alleles are therefore less capable to clear cells from toxic compounds. Both, transport and metabolism are affected in a gene-dose dependant manner. According to the number of low expression-related alleles of the respective transport protein, individuals can be classified as having either extensive (ET), intermediate (IT) or poor transporter capacity (PT) of the respective gene.

By genetic testing prior to onset of treatment with irinotecan, the MDR1- and MRP1 -related transport capacity of the patients can be predicted. The individual risk to adverse effects depends on the number of PM and/or PT alleles Individuals with PM-related alleles of CYP3A5 and UGT1A1 and PT-related alleles of MDR1 and MRP1 are at the highest risk to suffer from irinotecan toxicity.

Based on this knowledge, the initial dose can be adjusted prior to the first dose as shown by Brockmöller et al. (2000, Pharmacogenomics 1:125) for substrate drugs of CYP2D6, CYP2C9, and CYP2C19.

Dose adjustment can be achieved using a scoring system. For each PM- or PT-related allele a certain score is assigned e.g. a score of 2 is assigned to UGT1A1 PM alleles 226A, (SEQ ID NOs 9, 10, 11, 12, 540, 541) and 701A (SEQ ID NOs. 25, 26, 27, 28, 554, 555), and a score of 1 is assigned to the CYP3A5 PM-related alleles (47523T plus 35649A plus 145601T plus 145929A, 47523T plus 35649G plus 145601G plus 145929G, and 47523C plus 35649A plus 145601T plus 145929A), to the MDR1 low expression allele 176T (SEQ ID NOs.: 417, 418, 419, and 420), to the MRP1 low expression alleles 150727A (SEQ ID NOs. 217, 218, 219, and 220) and 259G (SEQ ID NOs. 277, 278, 279, and 280), to the MRP1 150727A allele (SEQ ID NOs. 217, 218, 219, and 220). After genotyping the scores are summarized and irinotecan dosage is adjusted according to the sum. Each single score corresponds to a dose reduction of 10%, i.e. a score of one corresponds to a 10% dose reduction, a score of two to 20%, a score of 3 to 30%, etc.

### EXAMPLE 4

### Culture Conditions and Biological Assays

The human epithelial cervical cancer cell line KB 3-1 with two subclones (KB 3-1 (MDR1⁺⁺⁺) and KB 3-1 (MDR1⁺⁺⁺, CYP3A5)) and the bladder cancer cell line RT112, also with subclone (RT112 (MDR1⁺, UGT1A1)), were cultured in Dulbecco's Modified Eagle Medium (DMEM) including 3.7 g/l NaHCO₃, 4.5 g/l D-Glucose, 1.028 g/l N-Acetyl-L-Alanyl-L-glutamine and supplemented with 10% fetal bovine, 1 mM Na-pyruvate and 1% non-essential amino acids. The human colon cancer cell line LS174T was cultured in Dulbecco's modified Eagle medium containing L-glutamine, pyridoxine hydrochloride and 25 mM Hepes buffer without phenol red, supplemented with 10% fetal bovine, 1 mM Na-pyruvate and 1% non-essential amino acids. All cells were incubated at 37° C. with 5% CO₂ in a humidified atmosphere.

Drugs

Irinotecan (CPT-11) and its active metabolite SN-38 were provided by Pharmacia. For preparation of stock solutions the substances were dissolved in methanol, 10 mg/ml for CPT-11 and 1 mg/ml for SN-38 and stored at 4° C. protected from light. Lower concentrated dilutions were prepared in PBS and cell culture medium. R-Verapamil was applied from SIGMA, dissolved in DMSO to 50 mg/ml and further diluted in PBS.

Treatment of Cells with Drugs

Cells were seeded in 96-well culture plates 24 h prior to treatment. With respect to differential growth rates KB 3-1 and RT112 cells were seeded at 700 cells/well, RT112 (MDR1⁺, UGT1A1) at 1000 cells/well and KB 3-1 (MDR1⁺⁺⁺) and KB 3-1 (MDR1⁺⁺⁺, CYP3A5) at 1200 cells/well. LS174T were seeded at 1.0×10⁴ cells/well. Cells were treated with freshly prepared serial dilutions in culture medium, 0, 0.5, 1, 2.5, 5, 7.5, 10, 25, 50, 75, 100 and 200 μg/ml for CPT-11, and 0, 0.1, 0.25, 0.5, 1, 5, 10, 25, 50, 75, 100 and 200 ng/ml for SN-38. Four well were treated with the same drug dilution. Cells were incubated for 3 days at 37° C. in a humidified 5% CO₂ atmosphere.

For MDR1 inhibition experiments R-Verapamil was added to 10 μg/ml final concentration in two wells of each drug dilution.

Cytotoxicity Assay

A commercially available MTS assay system (Promega, Madison, USA) was used to determine growth inhibition and cell death according to the instructions of the manufacturer. Three days after adding the drugs, 20 μl of the combined MTS/PMS solution was added to each well of the 96-well culture plate. The plate was incubated for at least 45 min at 37° C. in a humidified 5% CO₂ atmosphere and the absorbance at 492 nm was measured. The absorbance values of untreated control cells on each plate were set as 100% growth and used to calculate the remaining growth of drug treated cells. Untreated cells on the culture plates served as controls for unaffected growth and survival.

The drug concentration effecting a 50% inhibition of cell growth was defined as the IC₅₀.

RNA Preparation and cDNA Synthesis

From each cell batch used in these experiments messenger RNA was isolated from cell lysates by oligo-dT magnet beads (μMACS mRNA Isolation Kit; Miltenyi Biotech) following the instructions of the manufacturer. 250 ng mRNA of each cell line was applied in a 20 μl cDNA synthesis reaction with Superscript II reverse transcriptase (Gibco BRL). Dilutions of this cDNAs served as template in transcript specific amplification reactions.

PCR Primers and Reaction Conditions

PCRs were set up in 25 μl reactions with 0.5 units Taq Polymerase (Qiagen), 200 μM nucleotide mix, 5 μl cDNA template dilution and 0.2 μM gene specific primers, as indicated in Table 3. All reactions were run under the same amplification conditions, differing only in number of cycles (table ), 2 min pre-denaturation at 94° C., than for amplification: 45 sec denaturation at 94° C., 45 sec annealing at 62° C. and 45 sec elongation at 72° C., except for UGT1A1 which needed longer elongation of 2 min.

TABLE 3

Sequences of gene specific primers and conditions
for PCR reactions. F: forward primer; R: reverse
primer for mRNA sequences.

| | |
|---|---|
| cDNA | cycle |

| | | | |
|---|---|---|---|
| Gene | Primer sequence (5′-3′) | dilution | number |

| | | | |
|---|---|---|---|
| MDR1 | F: TGCCTTCATCGAGTCACTGCC | 1:100 | 26 |
| | R: TCACTGGCGCTTTGTTCGAGC | | |
| MRP1 | F: TCTCCAAGGAGCTGGACACA | 1:10 | 30 |
| | R: CGTGGTGACCTGCAATGAGT | | |
| UGT1A | F: GATGATGCCCTTGTTTGGTG | 1:100 | 30 |
| | R: TGTTTCAAGTTTGGAAATGACTAGGG | | |
| UGT1A1 | F: AACCTCTGGCAGGAGCAAAGG | 1:10 | 34 |
| | R: TGTTTTCAAGTTTGGAAATGACTAGGG | | |
| CYP3A4 | F: TCAGCCTGGTGCTGCTCTATCTAT | 1:10 | 34 |
| | R: AAGCCCTTATGGTAGGACAAAATATTT | | |
| CYP3A5 | F: TTGTTGGGAAATGTTTTGTCCTATC | 1:10 | 34 |
| | R: ACAGGGAGTTGACCTTCATACGTT | | |
| PLA2 | F: GCTGGTTCAGAAGGCCAAAC | 1:100 | 26 |
| (house keeping | R: GGGCCAGACCCAGTCTGATA | | |
| gene) | | | |

### EXAMPLE 5

### Expression of Genes Involved in Irinotecan Metabolism

Messenger RNA was isolated from the human bladder cancer cell line RT112, its subclone RT112 (MDR1, UGT1A1), the human epithelial cervical cancer cell line KB 3-1 and two subclones KB 3-1 (MDR1⁺⁺⁺) and KB 3-1 (MDR1⁺⁺⁺, CYP3A5), and the colon carcinoma cell line LS174T (ATCC CL-188). These mRNAs were reverse transcribed into cDNA and applied as templates in transcript-specific amplification reactions to determine the expression levels of genes involved in irinotecan transport and metabolism (MDR1, MRP1, UGT1A, UGT1A1, CYP3A4, CYP3A5). Amplification of the house keeping gene phospholipase A2 (PLA2) was used as a control for comparable cDNA amounts in the reactions.

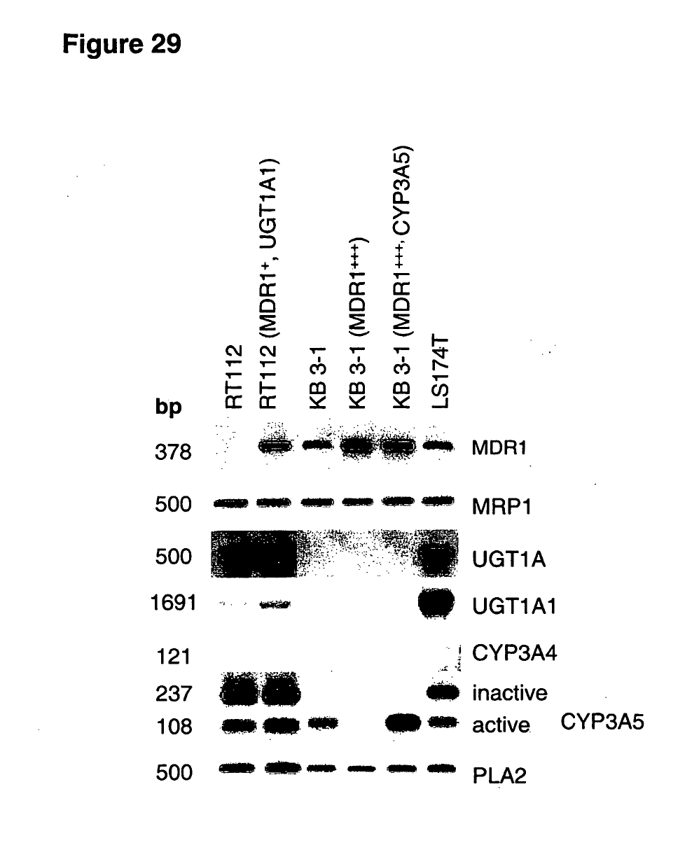

The amplification reactions in FIG. 29 show that the carcinoma cell lines RT112, KB 3-1, and LS174T have no or very low expression of MDR1, respectively. RT112 (MDR1, UGT1A1) is a subclone of RT112, which was selected for resistance to cytotoxic drugs as described in Seemann et al. (Urol Res 1995; 22:353-360), and is characterised by a moderately increased MDR1 expression. The drug resistant subclones KB 3-1 (MDR1⁺⁺⁺) and KB 3-1 (MDR1⁺⁺⁺, CYP3A5) were derived similarly from the original KB 3-1 cell line by exposure to MDR1 substrates. These subclones are characterized by highly increased MDR1 expression. They show >20-times more transcripts than the original KB 3-1 cells, implicating a very high MDR1 activity. MRP1 is expressed at the same level in all cell lines. Transcripts of UGT1A enzymes are present only in RT112, RT112 (MDR1, UGT1A1), and LS174T cells. UGT1A1 is only weakly expressed in RT112, stronger expressed in RT112 (MDR1, UGT1A1) and shows highest expression in LS174T cells. CYP3A4 was solely detected in very small amounts in LS174T. RT112 cells, RT112 (MDR1, UGT1A1), and LS174T show a heterozygous expression of the functionally inactive splice variant and the functionally active transcript of CYP3A5. In contrast, KB 3-1 and KB 3-1 (MDR1⁺⁺⁺) cells have only the active CYP3A5 transcript and the KB 3-1 (MDR1⁺⁺⁺, CYP3A5) showed the highest expression of the active CYP3A5 transcript, implicating that the latter have the highest CYP3A5 activity.

### EXAMPLE 6

### Colon and Other Epidermal Cancer Cell Lines with No or Low MDR1 and CYP3A5 Activity Are Sensitive to CPT-11 and SN-38

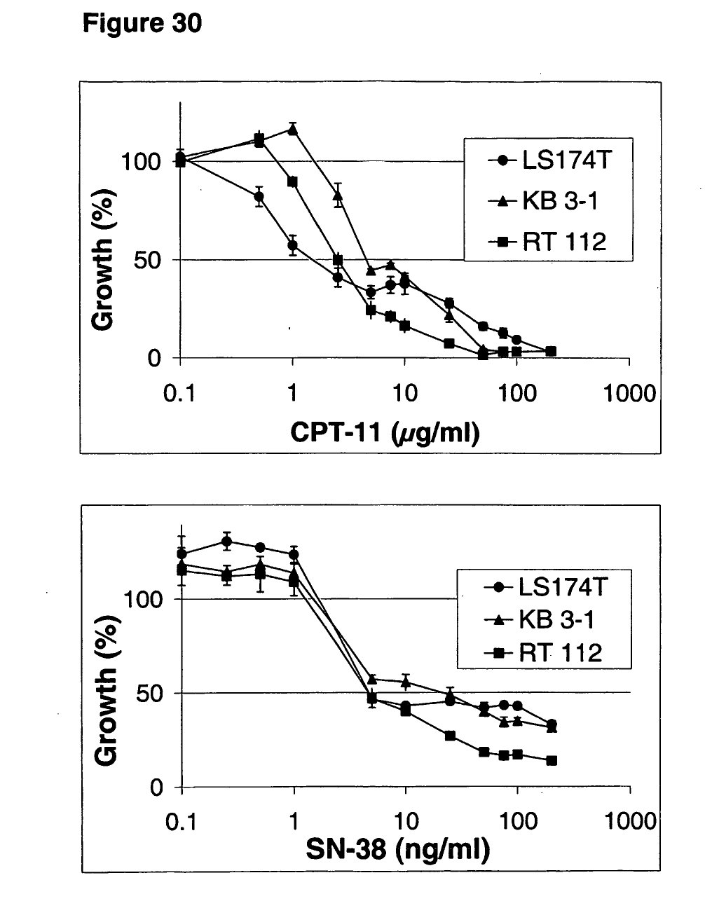

The colon cancer cell line LS174T, the cervical cancer cell line KB 3-1 and the bladder cancer cell line RT112 were seeded in 96-well culture plates 24 h prior to treatment. Four wells of each cell line were incubated with serial dilutions of CPT-11 and SN-38 and analysed as described above. FIG. 30 shows that all three epidermal cancer cell lines stop proliferation and die upon treatment with CPT-11 and SN-38. The concentrations resulting in 50% inhibition (IC₅₀) for CPT-11 are 1.5 μg/ml for LS174T, 2.5 μg/ml for RT112 and 5 μg/ml for KB 3-1 cells. The active metabolite of CPT-11, SN-38 shows a 1000-fold higher efficacy than CPT-11, since 10³-times lower concentrations cause the same degree of growth inhibition and cell death. The IC₅₀ of SN-38 is 5 ng/ml for LS174T cells, 4 ng/ml for RT112 cells and 25 ng/ml for KB 3-1 cells.

These results show that all three epidermal cancer cell lines although derived from different tissues are similarly sensitive to CPT-11 and SN-38 treatment. This also indicates that cancer cells expressing no or only low levels of MDR1 (FIG. 29) can be efficiently killed by CPT-11 and SN-38 (FIG. 30).

### EXAMPLE 7

### MDR1 Activity Correlates with Resistance of Cancer Cells Toward CPT-11 and SN-38

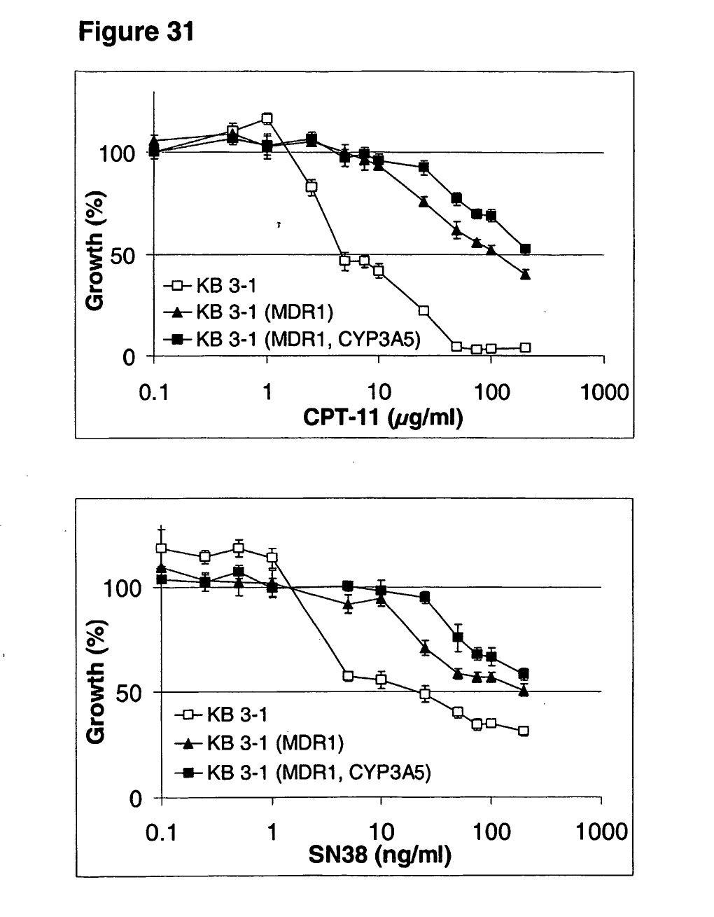

Cells of KB 3-1 and its strongly MDR1 expressing subclones KB 3-1 (MDR1⁺⁺⁺) and the KB 3-1 (MDR1⁺⁺⁺, CYP3A5) were seeded in 96-well culture 24 h prior to treatment. Four wells of each cell line were incubated with serial dilutions of CPT-11 and SN-38 and treated as described above. The inhibition curves (FIG. 31) of the MDR1 high expresser KB 3-1 subclones (KB 3-1 (MDR1⁺⁺⁺) and KB 3-1 (MDR1⁺⁺⁺, CYP3A5)) (FIG. 29) demonstrate a significant higher resistance to CPT-11 and SN-38 compared to the MDR1 low expresser KB 3-1 cell line (KB 3-1). The IC₅₀ for CPT-11 increases 17 to 40 told from 5 μg/ml in KB 3-1 to 85 μg/ml in KB 3-1 (MDR1⁺⁺⁺) and 200 μg/ml in KB 3-1 (MDR1⁺⁺⁺, CYP3A5) cells. The IC₅₀ for SN-38 increases at least 8 times from 25 ng/ml in KB 3-1 to 200 ng/ml in KB 3-1 (MDR1⁺⁺⁺) and >200 ng/ml in KB 3-1(MDR1⁺⁺⁺, CYP3A5).

CPT-11 and SN-38 are substrates of MDR1, and are therefore removed from the cells by MDR1 activity. The MDR1 expression level correlates inversely with the sensitivity of tumor cells towards CPT-11 and SN-38. Subsequently, the killing of cells with high MDR1 expresser phenotype requires much higher concentrations of CPT-11.

### EXAMPLE 8

### UGT1A1 Activity Correlates with Sensitivity Towards SN-38 and Not Towards CPT-11

CPT-11 and SN-38 sensitivity was compared between RT112 cells and its subclone RT112 (MDR1, UGT1A1). Four wells of each cell line were incubated with serial dilutions of CPT-11 and SN-38 and treated as described above.

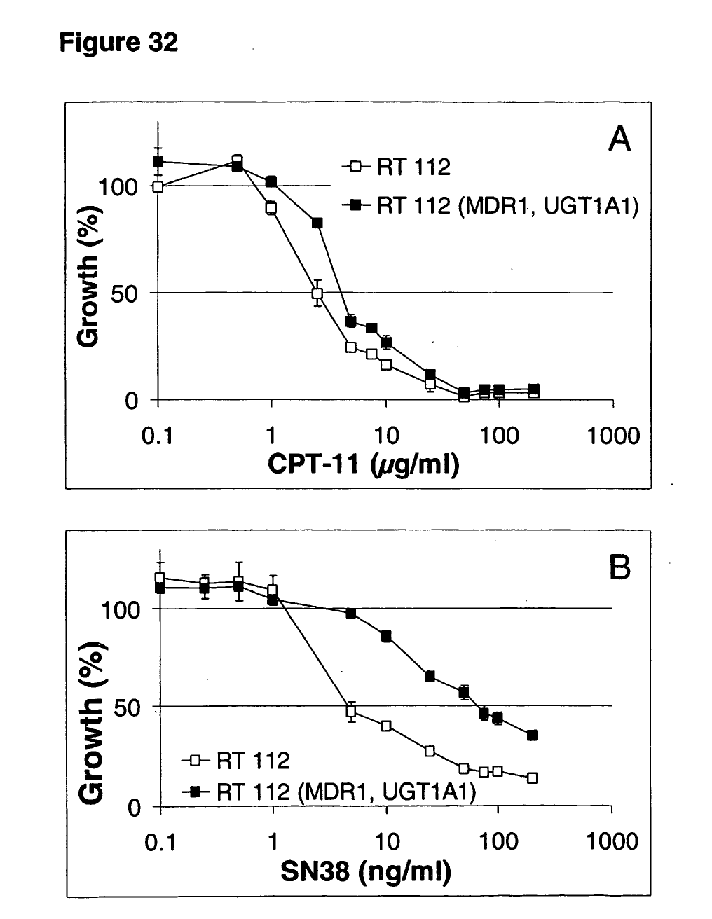

The difference in sensitivity against CPT-11 is only small as shown in FIG. 32A. The IC₅₀ of RT112(MDR1, UGT1A1) cells of 4 μg/ml CPT-11 is two-times higher compared to RT112 cells (IC₅₀ of 2.5 μg/ml). In contrast to RT112 cells which express no MDR1, RT112 MDR1, UGT1A1) cells express an intermediate amount of MDR1 which can explain the small though significant increase of CPT-11 sensitivity. A much stronger difference exists between RT112 (IC₅₀ of 4 ng/ml) and RT112 (MDR1, UGT1A1) cells (IC₅₀ of 75 ng/ml) after treatment with SN-38 (FIG. 32B). This 19-fold higher resistance of the RT112 (MDR1, UGT1A1) cell line can be explained by the additional detoxifying effect of UGT1A1 which is expressed at a higher level in RT112 (MDR1, UGT1A1) than in RT112 cells (FIG. 29). In contrast to SN-38, CPT-11 is not metabolized by UGTs. Therefore, CPT-11-related toxicity is not affected by UGT1A1 expression and the resistance-enhancing capabilitiy of UGTs in RT112(MDR1, UGT1A1) cells is only detected by application of SN-38.

### EXAMPLE 9

### MDR1 Inhibition Serves as Sensitizer Towards CPT-11 and SN-38 in MDR1 High Expressing But Not Low Expressing Cancer Cells

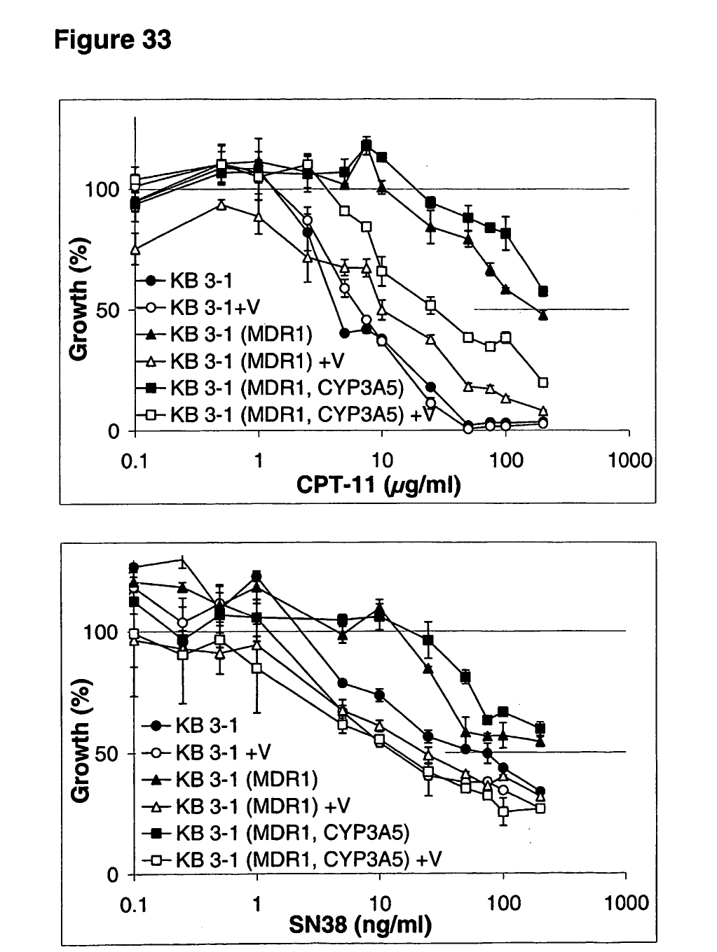

The sensitivity of KB 3-1 cells and its subclones KB 3-1 (MDR1⁺⁺⁺) and KB 3-1 (MDR1⁺⁺⁺, CYP3A5) against CPT-11 and SN-38 was assessed after blocking MDR1 function using the specific inhibitor R-Verapamil. Four wells of each cell line were incubated with serial dilutions of CPT-11, SN-38 and analysed as described above. Two wells were additionally treated with the MDR1 inhibitor R-Verapamil. FIG. 33 shows that addition of R-Verapamil has only marginal effects on the CPT-11 and SN-38 sensitivity of MDR1 low expresser KB 3-1 cells (CPT-11 and SN-38 IC50s of 5 μg/ml and 25 ng/ml without R-Verapamil versus 4.5 μg/ml and 15 ng/m with R-Verapamil, respectively). In contrast, the sensitivity of the MDR1 expressing cells KB 3-1(MDR1⁺⁺⁺) and KB 3-1(MDR1⁺⁺⁺, CYP3A5) towards CPT-11 and SN-38 was 8-fold and 10-fold higher after inhibition of MDR1 transport function with R-Verapamil. The IC₅₀ of KB 3-1 (MDR1⁺⁺⁺) cells for CPT-11 decreased from 85 μg/ml without to 10 μg/ml with R-Verapamil and from 200 μg/ml without to 25 μg/ml with R-Verapamil in KB 3-1 (MDR1⁺⁺⁺, CYP3A5) cells. The effect of MDR1 inhibition during SN-38 treatment is even stronger in these MDR1 high expresser cells, R-Verapamil blocked the MDR1 transport completely and they become as sensitive as KB 3-1 cells.

These results demonstrate that the MDR1 activity is relevant for resistance of cancer cells to CPT-11 and SN-38 and that inhibition of MDR1 sensitises the cells, so that they are more efficiently killed at lower drug concentrations.

### EXAMPLE 10

### CYP3A5 Activity Influences Resistance to CPT-11

KB 3-1 (MDR1⁺⁺⁺) and KB 3-1 (MDR1⁺⁺⁺, CYP3A5) cells which differ by their amounts of CYP3A5 (FIG. 29). Four wells of each cell line were incubated with serial dilutions of CPT-11, SN-38 and analyzed as described above. Two wells were additionally treated with the MDR1 inhibitor R-Verapamil.

Because MDR1 activity is a major determinant of cellular sensitivity toward CPT11 and SN-38, the MDR1 activity in these MDR1 high expresser cell lines was completely blocked using an excess of the specific MDR1 inhibitor R-Verapamil to analyze the impact of CYP3A5 on CPT-11 and SN-38 sensitivity without interference of MDR1.

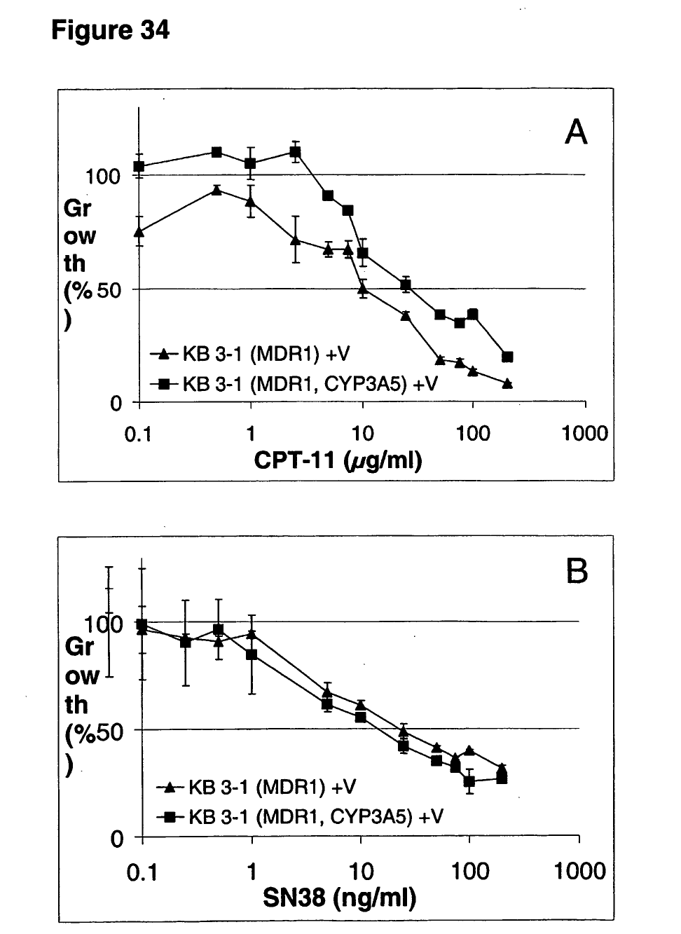

The high CYP3A5 expresser cell line KB 3-1 (MDR1⁺⁺, CYP3A5) is with an IC₅₀ of 25 μg/ml 2.5-times more resistant to CPT-11 than KB 3-1 (MDR1⁺⁺⁺) showing an IC₅₀ of 10 μg/ml (FIG. 34). No difference between these two cell lines can be observed regarding their sensitivity towards SN-38.

These experiments demonstrate a significant impact of CYP3A5 expression on the resistance to CPT-11 in contrast to SN-38. The fact that CYP3A5 activity had no influence on SN-38 toxicity further confirms the CYP3A5 effect, because CPT-11 but not SN-38 is metabolized by CYP3A5.

### EXAMPLE 11

### MDR1 Genotyping Improves Therapeutic Efficacy of Irinotecan by Genotype-based Prediction and Monitoring of Drug Resistance

Therapeutic efficacy and adverse effects of irinotecan depend on plasma levels and on intracellular tumor concentrations of the parent compound and the active metabolites (e.g. SN-38). The MDR1 gene controls the PGP-dependent penetration of irinotecan across membranes [Luo et al., Drug Metab Dispos 2002, 30:763-770; Jansen et al., Br J Cancer 1998, 77:359-65 Chu et al., J Pharmacol Exp Ther 1999; 288, 735-41; Sugiyama et al., Cancer Chemother Pharmacol 1998, 42 Suppl:S44-9] and is therefore an important determinant for its systemic availability and intracellular accumulation. The 176C>T nucleotide substitution (SEQ ID NOs. 217, 218, 219, and 220) of the MDR1 gene (Accession No: M29445) is associated with low PGP expression-related low drug efflux and patient carrying this substitution are more likely to respond to irinotecan treatment for two reasons: 1) Due to the lower amount of PGP in enterocytes more irinotecan can enter the body across the intestinal barrier causing more irinotecan to reach its site of action, the tumor. 2) Due to the lower amount of PGP in the tumor cell membranes more irinotecan can penetrate into the tumor cells to deploy its cytotoxic effects. The currently used standard dose of irinotecan kills highly effective most tumor cells within the first cycles of chemotherapy with only very few surviving drug-resistant tumor cells and tolerable adverse events. Independently from the mechanisms of drug resistance, in these patients, the number of surviving cells is to small to develop into a drug-resistant tumor which does not respond any longer to irinotecan therapy.

Patients with the high expresser MDR1 genotype (nucleotide C at position 176 of the MDR1 gene, Accession No: M29445) are less likely to respond to irinotecan treatment. Higher doses would be necessary to achieve a sufficiently efficient killing of tumor cells in order to prevent the development of a drug-resistant tumor. However, elevation of irinotecan dosage is limited due to the occurrence of intolerable adverse events (e.g. diarrhea, neutropenia, or thromboembolic complications). Alternatively, efficacy of irinotecan treatment can be improved by addition of a PGP inhibitor. A PGP inhibitor blocks efficiently the PGP function in MDR1 high expresser patients in such a way as to enable irinotecan to concentrate in the tumor cells for exerting its cytotoxicity as effective as in MDR1 low expresser patients. Consequently, genotypically MDR1 high expresser patients become phenotypically comparable to MDR1 low expressers.

According to the number of low or high expresser alleles of the MDR1 gene, individuals can be classified as having either extensive (ET, two high expresser alleles), intermediate (IT, one high expresser, one low expresser allele) or poor transport capacity (PT, two low expresser alleles). By genetic testing prior to onset of treatment with irinotecan, patients can be classified as ET, IT, or PT and the MDR1 -related transport capacity of the patients can be predicted. The individual risk of an insufficient anticancer treatment increases with the number of MDR1 high expresser alleles. Individuals with ET genotype are at the highest risk to suffer from insufficient response to irinotecan and are at the highest risk to develop a drug resistant tumor. ET patients should be treated with a PGP-inhibitor in addition to irinotecan and more closely monitored for adverse events and for the development of chemotherapy-related drug-resistance. Furthermore, these patients, who are at high risk for developing a drug-resistant tumor, can particularly benefit from taking a tumor biopsy between each cycle of chemotherapy with subsequent individual profiling of tumor cells for drug resistance.

1. A method of using irinotecan to treat a patient suffering from cancer which comprises:
(1) determining if the patient has one or more variant alleles of the MRP1 gene in the cancerous tissue;
(2) in a patient having one or more of such variant alleles, administering to the patient an amount of irinotecan which is sufficient to treat a patient having such variant alleles which amount is increased or decreased in comparison to the amount that is administered without regard to the patient's alleles in the MRP1 gene.

2. The method of claim 1, wherein the cancer is colorectal cancer, cervical cancer, gastric cancer, lung cancer, malignant glioma, ovarian cancer, or pancreatic cancer.

3. The method of claim 2 in which:
(1) the one or more variant alleles result in the patient expressing low amounts of the MRP1 gene product, whereby the amount of irinotecan administered to the patient is decreased to avoid toxicity; or
(2) the one or more variant alleles result in the patient expressing high amounts of the MRP1 gene product, whereby the amount of irinotecan administered to the patient is increased to enhance efficacy.

4. The method of claim 3, wherein the one or more variant alleles are in the promoter region of the MRP1 gene.

5. The method of claim 3, wherein the one or more variant alleles are in the coding region of the MRP1 gene.

6. The method of claim 3, wherein the one or more variant alleles are not in either the promoter region or the coding region of the MRP1 gene.

7. The method of claim 3, wherein the one or more variant alleles are in both the promoter region and the coding region of the MRP1 gene.

8. The method of claim 3, wherein the one or more variant alleles comprises a polynucleotide selected from the group consisting of:
(a) a polynucleotide having the nucleic acid sequence of any one of SEQ ID NOs:169, 170, 173, 174, 177, 178, 181, 182, 185, 186, 189, 190, 193, 194, 197, 198, 201, 202, 205, 206, 209, 210, 213, 214, 217, 218, 221, 222, 225, 226, 229, 230, 233, 234, 237, 238, 241, 242, 245, 246, 249, 250, 253, 254, 257, 258, 261, 262, 265, 266, 269, 270, 273, 274, 277, 278, 281, 282, 285, 286, 289, 290, 293, 294, 297, 298, 301, 302, 305, 306, 309, 310, 313, 314, 317, 318, 321, 322, 325, 326, 329, 330, 333 and/or 334;
(b) a polynucleotide encoding a polypeptide having the amino acid sequence of any one of SEQ ID NOs: 600, 602 and/or 604;
(c) a polynucleotide capable of hybridizing to a Multidrug Resistance Protein 1 (MRP1) gene, wherein said polynucleotide is having at a position corresponding to positions 57998, 57853, 53282, and/or 39508 of the MRP1 gene (Accession No: GI:7209451), a substitution or deletion of at least one nucleotide or at a position corresponding to positions 137667, 137647, 137710, 124667, and/or 38646 of the MRP1 gene (Accession No: AC026452), a substitution or deletion of at least one nucleotide or at a position corresponding to positions 27258, 27159, 34218, 34215, 55472, and/or 34206 to 34207 of the MRP1 gene (Accession No: AC003026), a substitution or deletion of at least one nucleotide or at a position corresponding to positions 21133, 14008, 18067, 17970, 17900, and/or 18195 of the MRP1 gene (Accession No: U91318), a substitution or deletion of at least one nucleotide or at a position corresponding to positions 79, 88, and/or 249 of the MRP1 gene (Accession No: AF022830), a substitution or deletion of at least one nucleotide or at a position corresponding to positions 95 and/or 259 of the MRP1 gene (Accession No: AF022831), a substitution or deletion of at least one nucleotide or at a position corresponding to positions 150727 and/or 33551 of the MRP1 gene (Accession No: AC025277), a substitution or deletion of at least one nucleotide or at a position corresponding to position 174 of the MRP1 gene (Accession No: AF022828), a substitution or deletion of at least one nucleotide or at a position corresponding to positions 248 and/or 258 of the MRP1 gene (Accession No: AF022829), a substitution or deletion of at least one nucleotide or at a position corresponding to positions 1884, 1625, 1163, 381, 233, 189, 440, and/or 1720 to 1723 of the MRP1 gene (Accession No: U07050), a substitution or deletion of at least one nucleotide or at a position corresponding to positions 926927 and/or 437/438 of the MRP1 gene (Accession No: U07050) a insertion of at least one nucleotide or at a position corresponding to position 55156/55157 of the MRP1 gene (Accession No: AC003026) a insertion of at least one nucleotide;
(d) a polynucleotide capable of hybridizing to a MRP1 gene, wherein said polynucleotide is having at a position corresponding to position 21133, 14008 and/or 18195 of the MRP1 gene (Accession No: U91318) or at a position corresponding to position 27258 and/or 34218 of the MRP1 gene (Accession No: AC003026) or at a position corresponding to position 79 of the MRP1 gene (Accession No: AF022830) or at a position corresponding to position 57998, and/or 57853 of the MRP1 gene (Accession No: GI:7209451) or at a position corresponding to position 137667 and/or 137647 of the MRP1 gene (Accession No: AC026452) or at a position corresponding to position 150727 and/or 33551 of the MRP1 gene (Accession No: AC025277) or at a position corresponding to position 248 of the MRP1 gene (Accession No: AF022829) or at a position corresponding to position 1884, 1625, 233, and/or 189 of the MRP1 gene (Accession No: U07050) an A, at a position corresponding to position 39508 of the MRP1 gene (Accession No: GI:7209451) or at a position corresponding to position 17900, 18067 and/or 18195 of the MRP1 gene (Accession No: U91318) or at a position corresponding to position 174 of the MRP1 gene (Accession No: AF022828) or at a position corresponding to position 440 and/or 1163 of the MRP1 gene (Accession No: U07050) a T, at a position corresponding to position 88 of the MRP1 gene (Accession No: AF022830) or at a position corresponding to position 95 of the MRP1 gene (Accession No: AF022831) or at a position corresponding to position 27159, 55472 and/or 34215 of the MRP1 gene (Accession No: AC003026) or at a position corresponding to position 124667 and/or 38646 of the MRP1 gene (Accession No: AC026452) or at a position corresponding to position 53282 of the MRP1 gene (Accession No: GI:7209451) or at a position corresponding to position 137710 of the MRP1 gene (Accession No: AC026452) a C, at a position corresponding to position 249 of the MRP1 gene (Accession No: AF022830) or at a position corresponding to position 258 of the MRP1 gene (Accession No: AF022829) or at a position corresponding to position 259 of the MRP1 gene (Accession No: AF022831) or at a position corresponding to position 381 of the MRP1 gene (Accession No: U07050) a G, at a position corresponding to position 17970 of the MRP1 gene (Accession No: U91318) a deletion of a T or at a position corresponding to position 34206 to 34207 of the MRP1 gene (Accession No: AC003026) a deletion of a AT or at a position corresponding to position 1720 to 1723 of the MRP1 gene (Accession No: U07050) a deletion of GGTA, at a position corresponding to position 926/927 a insertion of a T and/or 437/438 of the MRP1 gene (Accession No: U07050) a insertion of a TCCTTCC, at a position corresponding to position 55156/55157 of the MRP1 gene (Accession No: AC003026) a insertion of TGGGGC;
(e) a polynucleotide encoding an MRP1 polypeptide or fragment thereof, wherein said polypeptide comprises an amino acid substitution at a position corresponding to positions 600, 602, and/or 604 of the MRP1 polypeptide (Accession No: G2828206);
(f) a polynucleotide encoding an MRP1 polypeptide or fragment thereof, wherein said polypeptide comprises an amino acid substitution of Phe to Cys at a position corresponding to position 239 of the MRP1 polypeptide (Accession No: G2828206) or/and Arg to Ser at a position corresponding to position 433 of the MRP1 polypeptide (Accession No: G2828206) or/and Arg to Gin at a position corresponding to position 723 of the MRP1 polypeptide (Accession No: G2828206).

9. The method of claim 8, wherein the one or more variant alleles comprises a polynucleotide selected from the group consisting of:
(a) a polynucleotide having the nucleic acid sequence of any one of SEQ ID NO: 181, 209, 217, 205, 277, 281, 301, 325, 229, 193, 313, 293 or 253;
(b) a polynucleotid encoding a polypeptide having the amino acid sequence of SEQ ID NO: 600;
(c) a polynucleotide capable of hybridizing to a MRP1 gene, wherein said polynucleotide is having a substitution at a position corresponding to position 137647 of the MRP1 gene (Accession No: AC026452), 95 of the MRP1 gene (Accession No: AF022831), 53282 of the MRP1 gene (Accession No: GI:7209451), 249 of the MRP1 gene (Accession No: AF022830), 259 of the MRP1 gene (Accession No: AF022831), 124667 of the MRP1 gene (Accession No: AC026452), 381, 440, 1625 of the MRP1 gene (Accession No: U07050), 34218 of the MRP1 gene (Accession No: AC003026), 18067 or 17900 of the MRP1 gene (Accession No: U91318) or an insertion of at least one nucleotide at a position corresponding to position 926/927 of the MRP1 gene (Accession No: U07050);
(d) a polynucleotide capable of hybridizing to a MRP1 gene, wherein said polynucleotide is having a T at a position corresponding to position 137647 of the MRP1 gene (Accession No: AC026452), 18067 or 17900 of the MRP1 gene (Accession No: U91318), 440 of the MRP1 gene (Accession No: U07050), a C at a position corresponding toposition 95 of the MRP1 gene (Accession No: AF022831),124667 of the MRP1 gene (Accession No: AC026452), a G at a position corresponding to position 53282 of the MRP1 gene (Accession No: GI:7209451), 249 of the MRP1 gene (Accession No: AF022830), 259 of the MRP1 gene (Accession No: AF022831), 381 of the MRP1 gene (Accession No: U07050), or an A at a position corresponding to position 34218 of the MRP1 gene (Accession No: AC003026) or 1625 of the MRP1 gene (Accession No: U07050) or an insertion of a T at a position corresponding to position 926/927 of the MRP1 gene (Accession No: U07050);
(e) a polynucleotide encoding an MRP1 polypeptide or fragment thereof, wherein said polypeptide comprises an amino acid substitution at a position corresponding to position 329 of the MRP1 polypeptide (Accession No: G2828206); and
(d) a polynucleotide encoding an MRP1 polypeptide or fragment thereof, wherein said polypeptide comprises an amino acid substitution of Phe to Cys at a position corresponding to position 329 of the MRP1 polypeptide (Accession No: G2828206).

10. The method of claim 8, in which the one or more variant alleles results in the patient expressing low amounts of the MRP1 gene product, whereby the amount of ironotecan administered to the patient is decreased.

11. The method of claim 8, in which the one or more variant alleles results in the patient expressing high amounts of the MRP1 gene product, whereby the amount of irinotecan administered to the patient is increased.

12. The method of claim 9, in which the one or more variant alleles results in the patient expressing low amounts of the MRP1 gene product, whereby the amount of irinotecan administered to the patient is decreased.

13. The method of claim 9, in which the one or more variant alleles results in the patient expressing high amounts of the MRP1 gene product, whereby the amount of irinotecan administered to the patient is increased.

14. A method for determining whether a patient is at risk for a toxic reaction to treatment with irinotecan which comprises determining if the patient has one or more variant alleles of the MRP1 gene.

15. The method of claim 14, which further comprises administering to the patient reduced amounts of irinotecan.

16. A method for determining the optimum treatment regimen for administering irinotecan to a patient suffering from cancer which comprises:
(1) determining if the patient has one or more variant alleles of the MRP1 gene;
(2) in a patient having one or more of such alleles increasing or decreasing the amount of irinotecan in comparison to the amount that is administered without regard to the patient's alleles in the MRP1 gene.

17. A method of treating cancer in a patient having one or more variant alleles of the MRP1 gene such that expression levels of the MRP1 gene product are lower than in the general population and so indicates high sensitivity to irinotecan which comprises administering to the patient a decreased amount of irinotecan.

18. A method of treating cancer in a patient having one or more variant alleles of the MRP1 gene such that expression levels of the MRP1 gene product are higher than in the and so indicates resistance or predisposition to resistance to irinotecan which comprises administering to the patient an increased amount of irinotecan.

19. The method of claim 18, in which patients that have a variant allele that indicates resistance or predisposition to resistance are treated with an MRP1 inhibitor.

20. The method of claim 19, wherein the MRP1 inhibitor is selected from the group consisting of SDZ-PSC 833, SDZ 280-446, MK571, MS209(quinolone derivative), PAK-104p, Verapamil, Benzbromarone, Dipyridamole, Furosemide, Gamma-GS(naphtyl)cysteinyl-glycine diethyl ester, Genistein, Quinidine, Rifampicin, RU 486, Sulfinpyrazone.

21. The method of claim 17, which further comprises monitoring the patient during treatment by assaying for changes in expression levels of the MRP1 gene product in the cancerous cells whereby an increase in the expression level of the MRP1 gene product is compensated for by an increase in the amount of irinotecan administered to the patient.

22. A method of treating cancer in a patient which comprises internally administering to the patient an effective amount of irinotecan, wherein the treatment regimen is modified based upon the genotype of the patient's MRP1 gene.

23. A method of treating a population of patients suffering from cancer which comprises:
(1) determining, on a patient by patient basis, if the patient has one or more variant alleles of the MRP1 gene;
(2) in a patient having one or more of such variant alleles, administering to the patient an amount of irinotecan which is sufficient to treat a patient having such variant alleles which amount is increased or decreased in comparison to the amount that is administered without regard to the patient's alleles in the MRP1 gene.

24. A method for predicting sensitivity to irinotecan in a patient suffering from cancer which comprises determining if the patient has one or more variant alleles of the MRP1 gene, which alleles indicate that the cancerous cells express low or high amounts of the MRP1 gene product, whereby low expression indicates high sensitivity to irinotecan and high expression indicates resistance or predisposition to resistance to irinotecan.

25. The method of claim 24, in which patients that have a genotype that indicates resistance or predisposition to resistance are treated with a MRP1 inhibitor.

26. The method of claim 25, wherein the MRP1 inhibitor is selected from the group consisting of SDZ-PSC 833, SDZ 280-446, MK571, MS209 (quinolone derivative), PAK-104p, Verapamil, Benzbromarone, Dipyridamole, Furosemide, Gamma-GS(naphtyl)cysteinyl-glycine diethyl ester, Genistein, Quinidine, Rifampicin, RU 486, Sulfinpyrazone.

27. The method of claim 26, wherein the patients that have a genotype that indicates resistance or predisposition to resistance are monitored during treatment by assaying for expression levels of the MRP1 gene product in the cancerous cells.

28. Use of irinotecan or a derivative thereof for the preparation of a pharmaceutical composition for treating colorectal cancer, cervical cancer, gastric cancer, lung cancer, malignant glioma, ovarian cancer, and pancreatic cancer in a subject having a genome with a variant allele which comprises a polynucleotide selected from the group consisting of:
(a) a polynucleotide having the nucleic acid sequence of any one of SEQ ID NOs: 169, 170, 173, 174, 177, 178, 181, 182, 185, 186, 189, 190, 193, 194, 197, 198, 201, 202, 205, 206, 209, 210, 213, 214, 217, 218, 221, 222, 225, 226, 229, 230, 233, 234, 237, 238, 241, 242, 245, 246, 249, 250, 253, 254; 257, 258, 261, 262, 265, 266, 269, 270, 273, 274, 277, 278, 281, 282, 285, 286, 289, 290, 293, 294, 297, 298, 301, 302, 305, 306, 309, 310, 313, 314, 317, 318, 321, 322, 325, 326, 329, 330, 333 and/or 334;
(b) a polynucleotide encoding a polypeptide having the amino acid sequence of any one of SEQ ID NOs: 600, 602 and/or 604;
(c) a polynucleotide capable of hybridizing to a Multidrug Resistance Protein 1 (MRP1) gene, wherein said polynucleotide is having at a position corresponding to positions 57998, 57853, 53282, and/or 39508 of the MRP1 gene (Accession No: GI: 7209451), a substitution or deletion of at least one nucleotide or at a position corresponding to positions 137667, 137647, 137710, 124667, and/or 38646 of the MRP1 gene (Accession No: AC026452), a substitution or deletion of at least one nucleotide or at a position corresponding to positions 27258, 27159, 34218, 34215, 55472, and/or 34206 to 34207 of the MRP1 gene (Accession No: AC003026), a substitution or deletion of at least one nucleotide or at a position corresponding to positions 21133, 14008, 18067, 17970, 17900, and/or 18195 of the MRP1 gene (Accession No: U91318), a substitution or deletion of at least one nucleotide or at a position corresponding to positions 79, 88, and/or 249 of the MRP1 gene (Accession No: AF022830), a substitution or deletion of at least one nucleotide or at a position corresponding to positions 95 and/or 259 of the MRP1 gene (Accession No: AF02283 1), a substitution or deletion of at least one nucleotide or at a position corresponding to positions 150727 and/or 33551 of the MRP1 gene (Accession No: AC025277), a substitution or deletion of at least one nucleotide or at a position corresponding to position 174 of the MRP1 gene (Accession No: AF022828), a substitution or deletion of at least one nucleotide or at a position corresponding to positions 248 and/or 258 of the MRP1 gene (Accession No: AF022829), a substitution or deletion of at least one nucleotide or at a position corresponding to positions 1884, 1625, 1163, 381, 233, 189, 440, and/or 1720 to 1723 of the MRP1 gene (Accession No: U07050), a substitution or deletion of at least one nucleotide or at a position corresponding to positions 926927 and/or 437/438 of the MRP1 gene (Accession No: U07050) a insertion of at least one nucleotide or at a position corresponding to position 55156/55157 of the MRP1 gene (Accession No: AC003026) a insertion of at least one nucleotide;
(d) a polynucleotide capable of hybridizing to a MRP1 gene, wherein said polynucleotide is having at a position corresponding to position 21133, 14008 and/or 18195 of the MRP1 gene (Accession No: U91318) or at a position corresponding to position 27258 and/or 34218 of the MRP1 gene (Accession No: AC003026) or at a position corresponding to position 79 of the MRP1 gene (Accession No: AF022830) or at a position corresponding to position 57998, and/or 57853 of the MRP1 gene (Accession No: GI:7209451) or at a position corresponding to position 137667 and/or 137647 of the MRP1 gene (Accession No: AC026452) or at a position corresponding to position 150727 and/or 33551 of the MRP1 gene (Accession No: AC025277) or at a position corresponding to position 248 of the MRP1 gene (Accession No: AF022829) or at a position corresponding to position 1884, 1625, 233, and/or 189 of the MRP1 gene (Accession No: U07050) an A, at a position corresponding to position 39508 of the MRP1 gene (Accession No: GI:7209451) or at a position corresponding to position 17900, 18067 and/or 18195 of the MRP1 gene (Accession No: U91318) or at a position corresponding to position 174 of the MRP1 gene (Accession No: AF022828) or at a position corresponding to position 440 and/or 1163 of the MRP1 gene (Accession No: U07050) a T, at a position corresponding to position 88 of the MRP1 gene (Accession No: AF022830) or at a position corresponding to position 95 of the MRP1 gene (Accession No: AF02283 1) or at a position corresponding to position 27159, 55472 and/or 34215 of the MRP1 gene (Accession No: AC003026) or at a position corresponding to position 124667 and/or 38646 of the MRP1 gene (Accession No: AC026452) or at a position corresponding to position 53282 of the MRP1 gene (Accession No: GI:7209451) or at a position corresponding to position 137710 of the MRP1 gene (Accession No: AC026452) a C, at a position corresponding to position 249 of the MRP1 gene (Accession No: AF022830) or at a position corresponding to position 258 of the MRP1 gene (Accession No: AF022829) or at a position corresponding to position 259 of the MRP1 gene (Accession No: AF02283 1) or at a position corresponding to position 381 of the MRP1 gene (Accession No: U07050) a G, at a position corresponding to position 17970 of the MRP1 gene (Accession No: U91318) a deletion of a T or at a position corresponding to position 34206 to 34207 of the MRP1 gene (Accession No: AC003026) a deletion of a AT or at a position corresponding to position 1720 to 1723 of the MRP1 gene (Accession No: U07050) a deletion of GGTA, at a position corresponding to position 926/927 a insertion of a T and/or 437/438 of the MRP1 gene (Accession No: U07050) a insertion of a TCCTTCC, at a position corresponding to position 55156/55157 of the MRP1 gene (Accession No: AC003026) a insertion of TGGGGC;
(e) a polynucleotide encoding an MRP1 polypeptide or fragment thereof, wherein said polypeptide comprises an amino acid substitution at a position corresponding to positions 600, 602, and/or 604 of the MRP1 polypeptide (Accession No: G2828206);
(f) a polynucleotide encoding an MRP1 polypeptide or fragment thereof, wherein said polypeptide comprises an amino acid substitution of Phe to Cys at a position corresponding to position 239 of the MRP1 polypeptide (Accession No: G2828206) or/and Arg to Ser at a position corresponding to position 433 of the MRP1 polypeptide (Accession No: G2828206) or/and Arg to Gin at a position corresponding to position 723 of the MRP1 polypeptide (Accession No: G2828206).

29. The use of claim 28, wherein a nucleotide deletion, addition and/or substitution comprised by said polynucleotide results in an altered expression of the variant allele compared to the corresponding wild type alleles.

30. The use of claim 29, wherein said altered expression is decreased or increased expression.

31. The use of claim 28, wherein a nucleotide deletion, addition and/or substitution comprised by said polynucleotide results in an altered activity of the polypeptide encoded by the variant allele compared to the polypeptide encoded by the corresponding wild type allele.

32. The use of claim 31, wherein said altered activity is decreased or increased activity.

33. The use of claim 28, wherein said subject is an animal.

34. The use of claim 33, wherein said subject is a mouse.

35. The use of claim 28, wherein said subject is a human.

36. The use of claim 35, wherein said human is African or Asian.

37. A method for selecting a suitable therapy for a subject suffering from colorectal cancer, cervical cancer, gastric cancer, lung cancer, malignant glioma, ovarian cancer, and pancreatic cancer, wherein said method comprises:
(a) determining the presence or absence of a variant allele as specified in claim 28 in the genome of a subject in a sample obtained from said subject; and
(b) selecting a suitable therapy for said subject based on the results obtained in (a).
